(19) United States
(12) Patent Application Publication
(10) Pub. No.: US 2023/0377355 A1
(43) Pub. Date: Nov. 23, 2023

(54) SYNTHETIC POOLING FOR ENRICHING DISEASE SIGNATURES

(71) Applicant: New York Stem Cell Foundation, Inc., New York, NY (US)

(72) Inventors: Daniel John Paull, New York, NY (US); Bianca Migliori, New York, NY (US)

(21) Appl. No.: 18/320,694

(22) Filed: May 19, 2023

Related U.S. Application Data
(60) Provisional application No. 63/344,164, filed on May 20, 2022.

(51) Int. Cl. G06V 20/69; G06V 10/82; G06V 10/774; G06V 10/77; G06T 7/00; G16H 50/20; G06N 5/022

(52) U.S. Cl. CPC G06V 20/698; G06V 10/82; G06V 10/774; G06V 10/7715; G06T 7/0012; G16H 50/20; G06N 5/022; G06T 2207/30036; G06T 2207/20084; G06T 2207/20081; G06T 2207/10064

(57) ABSTRACT

The present disclosure provides automated methods and systems for implementing a pipeline involving the training and deployment of a predictive model for predicting cellular diseased state (e.g., neurodegenerative disease state such as presence or absence of Parkinson's Disease) and for identifying features specific to a disease. Such a predictive model is trained by using training data generated from at least one cohort of synthetically pooled cells of a known disease state.

### CROSS REFERENCE TO RELATED APPLICATIONS

The present application claims priority under 35 U.S.C. § 119 from U.S. provisional patent application Ser. No. 63/344,164, entitled “Synthetic Pooling for Enriching Disease Signatures,” filed May 20, 2022, the subject matter of which is incorporated herein by reference in its entirety.

### FIELD OF INVENTION

The present invention relates generally to the field of predictive analytics, and more specifically to automated methods and systems for predicting disease states and identifying phenotypes of specific diseases by synthetically pooling cells from different donors during model training.

### BACKGROUND OF THE INVENTION

Machine learning-based technology has been found to be a promising tool in early diagnosis and interpretation of medical images as well as discovery and development of new therapies. For example, new advancements in artificial intelligence (AI) and deep learning approaches have paved the way to accelerate therapeutic discovery specifically in drug repurposing, distinguishing cellular phenotypes, and elucidating mechanisms of action. In parallel, the use of large data sets such as high-content imaging has the ability to capture patient-specific patterns to glean insights into human pathology. Several works have reported the use of AI and large data sets to uncover disease phenotypes and biomarkers (Yang et al., 2019) (Teves et al., 2017), but the power of these studies is limited. One plausible theory is that high content imaging screens for identifying disease phenotypes suffer from high donor-specific variation, which tends to hide the features characterizing the disease, as the strongest distinctive signal is the patient-specific fingerprinting (Schiff et al., 2020).

### SUMMARY OF THE INVENTION

Disclosed herein are methods and systems for developing an automated high-throughput screening platform for predicting disease state of cells and for identifying disease-specific features. Disclosed herein is a method comprising: obtaining or having obtained one or more cells of a common state; capturing a plurality of images corresponding to the one or more cells; and analyzing the plurality of images using a predictive model to predict a presence or absence of a known disease state for the one or more cells, the predictive model trained to distinguish between morphological profiles of healthy cells and cells in a known disease state, where the predictive model is trained using training data generated from at least one cohort of synthetically pooled cells of the known disease state.

In various embodiments, the at least one cohort of synthetically pooled cells are randomly selected from different donors, and the predictive model is trained by averaging embeddings or fixed feature vectors of the pooled cells randomly selected from different donors, which causes donor-specific variations to be smoothened and disease-specific features to be highlighted when training the predictive model. In various embodiments, the predictive model more accurately distinguishes between the morphological profiles of healthy cells and cells in the known disease state in comparison to a predictive model that is trained without using a cohort of synthetically pooled cells. In various embodiments, the predictive model trained to distinguish between the morphological profiles of healthy cells and cells in the known disease state achieves an AUC of at least 0.95. In various embodiments, the predictive model trained to distinguish between the morphological profiles of healthy cells and cells in the known disease state achieves an accuracy of at least 0.88.

In various embodiments, the at least one cohort of synthetically pooled cells is built by randomly selecting a number of single cells or randomly selecting a number of tiles. In various embodiments, the synthetically pooled cells are formed by pooling together a plurality of cell lines of the known disease state or healthy state. In various embodiments, the plurality of cell lines are obtained from different subjects of the known disease state or healthy state. In various embodiments, pooling together the plurality of cell lines comprises combining embeddings or fixed feature vectors of randomly selected single cells. In various embodiments, combining the embeddings from the randomly selected single cells comprises averaging the embeddings or fixed feature vectors of the randomly selected single cells. In various embodiments, pooling together the plurality of cell lines does not involve physically pooling together the randomly selected single cells. In various embodiments, the at least one cohort of synthetically pooled cells are divided into separate training and testing folds for training the predictive model.

In various embodiments, the predictive model is trained by: capturing a plurality of images corresponding to the randomly selected single cells of the known disease state or healthy state; and using the plurality of images corresponding to the randomly selected single cells of the known disease state or healthy state to train the predictive model to distinguish between the morphological profiles of cells of the known disease state and cells of the healthy state. In various embodiments, using the plurality of images corresponding to the randomly selected single cells of the known disease state or healthy state to train the predictive model further comprises averaging embeddings of the plurality of images corresponding to the randomly selected single cells of the known disease state or healthy state. In various embodiments, the one or more cells of a common state comprise cells of a single cell line from a single subject. In various embodiments, analyzing the plurality of images for the one or more cells of a common state further comprises averaging embeddings from the one or more cells of a common state. In various embodiments, to distinguish between the morphological profiles of healthy cells and cells in the known disease state for the one or more cells of a common state, the predictive model is trained to compare an averaged embedding of the one or more cells of a common state to an averaged embedding of the plurality of images corresponding to the randomly selected single cells of the known disease state or healthy state.

In various embodiments, the predictive model is trained to predict the presence or absence of the known disease state with a prediction probability. In various embodiments, the healthy cells or the cells in the known disease state serve as a reference ground truth for training the predictive model. In various embodiments, the method further includes: prior to capturing the plurality of images corresponding to the one or more cells of a common state, providing a perturbation to the one or more cells of a common state, the perturbation causing the one or more cells from a known disease state to a unknown disease state; subsequent to analyzing the plurality of images of the one or more cells of a common state, comparing the predicted state of the one or more cells to the known disease state of the one or more cells known before providing the perturbation; and based on the comparison, identifying the perturbation as having one of a therapeutic effect, a detrimental effect, or no effect. In various embodiments, the predictive model is one of a neural network, random forest, or regression model. In various embodiments, the neural network is a multilayer perceptron model. In various embodiments, the regression model is one of a logistic regression model or a ridge regression model.

In various embodiments, each of the morphological profiles comprises values of imaging features or comprises a transformed representation of images that define a known disease state or a healthy state of a cell. In various embodiments, the imaging features comprise one or more of cell features. In various embodiments, the cell features comprise one or more of cellular shape, cellular size, cellular organelles, object-neighbors features, mass features, intensity features, quality features, texture features, and global features. In various embodiments, the cell features are determined via fluorescently labeled biomarkers. In various embodiments, the cell features are determined via fluorescently labeled biomarkers identifying one or more of cell nucleus, cell nucleoli, plasma membrane, cytoplasmic RNA, endoplasmic reticulum, actin, Golgi apparatus, and mitochondria. In various embodiments, each cell in the one or more cells of a common state is one of a stem cell, a partially differentiated cell, or a terminally differentiated cell.

In various embodiments, each cell in the one or more cells of a common state is a somatic cell. In various embodiments, the somatic cell is a fibroblast or a peripheral blood mononuclear cell (PBMC). In various embodiments, the one or more cells of a common state are obtained from a subject through a tissue biopsy or blood draw. In various embodiments, the tissue biopsy is obtained from an extremity of the subject. In various embodiments, the morphological profile is extracted from a layer of a deep learning neural network. In various embodiments, the morphological profile is an averaged embedding representing a dimensionally reduced representation of values of the layer of the deep learning neural network. In various embodiments, the layer of the deep learning neural network is a penultimate layer of the deep learning neural network.

In various embodiments, the method further includes: prior to capturing the plurality of images corresponding to the one or more cells of a common state, staining or having stained the one or more cells of a common state using one or more fluorescent dyes. In various embodiments, at least 5 cell features derive from fluorescently labeled biomarkers identifying plasma membrane. In various embodiments, at least 30 cell features derive from fluorescently labeled biomarkers identifying plasma membrane. In various embodiments, at least 5 cell features derive from fluorescently labeled biomarkers identifying cell nucleus. In various embodiments, at least 25 cell features derive from fluorescently labeled biomarkers identifying cell nucleus. In various embodiments, at least 5 cell features derive from fluorescently labeled biomarkers identifying endoplasmic reticulum. In various embodiments, at least 10 cell features derive from fluorescently labeled biomarkers identifying endoplasmic reticulum. In various embodiments, at least 5 cell features derive from fluorescently labeled biomarkers identifying mitochondria. In various embodiments, at least 35 cell features derive from fluorescently labeled biomarkers identifying mitochondria. In various embodiments, at least 5 cell features derive from fluorescently labeled biomarkers identifying RNA. In various embodiments, at least 10 cell features derive from fluorescently labeled biomarkers identifying RNA. In various embodiments, at least 60 correlated cell features derive from various fluorescence channels. In various embodiments, at least 20 correlated cell features derive from various fluorescence channels. In various embodiments, each of the plurality of images corresponding to the one or more cells of a common state corresponds to a fluorescent channel. In various embodiments, the steps of obtaining or having obtained the one or more cells of a common state and capturing the plurality of images corresponding to the one or more cells of a common state are performed in a high-throughput format using an automated array. In various embodiments, a common state is one of a common disease state, a common source, a common processing state, or a common growth state.

In various embodiments, the disease state of the cell predicted by the predictive model is a classification of at least two categories. In various embodiments, the at least two categories comprise a presence or absence of a neurodegenerative disease. In various embodiments, the at least two categories comprise a first subtype or a second subtype of a neurodegenerative disease. In various embodiments, the at least two categories further comprise a third subtype of the neurodegenerative disease. In various embodiments, the neurodegenerative disease is any one of Parkinson's Disease (PD), Alzheimer's Disease, Amyotrophic Lateral Sclerosis (ALS), Infantile Neuroaxonal Dystrophy (INAD), Multiple Sclerosis (MS), Amyotrophic Lateral Sclerosis (ALS), Batten Disease, Charcot-Marie-Tooth Disease (CMT), Autism, post-traumatic stress disorder (PTSD), schizophrenia, frontotemporal dementia (FTD), multiple system atrophy (MSA), and a synucleinopathy. In various embodiments, the first subtype comprises an LRRK2 subtype. In various embodiments, the second subtype comprises a sporadic PD subtype. In various embodiments, the third subtype comprises a GBA subtype.

In various embodiments, the method further includes: identifying a plurality of features associated with the known disease state when the one or more cells are predicted to be the known disease state; ranking the plurality of features according to a degree of difference of the features between the known disease state and the healthy state; and selecting a list of top-ranked features according to a predefined threshold. In various embodiments, the method further includes filtering the top-ranked features by removing a subset of features that are correlated; and updating the list of top-ranked features by excluding the subset of features, where the updated list of top-ranked features are designated as a phenotype for characterizing the known disease state.

Additionally disclosed herein is a non-transitory computer readable medium comprising instructions that, when executed by a processor, cause the processor to: capture a plurality of images corresponding to one or more cells of a common state; and analyze the plurality of images using a predictive model to predict a presence or absence of a known disease state for the one or more cells, the predictive model trained to distinguish between morphological profiles of healthy cells and cells in a known disease state, where the predictive model is trained using training data generated from at least one cohort of synthetically pooled cells of the known disease state.

In various embodiments, the predictive model more accurately distinguishes between the morphological profiles of healthy cells and cells in the known disease state in comparison to a predictive model that is trained without using a cohort of synthetically pooled cells. In various embodiments, the predictive model trained to distinguish between the morphological profiles of healthy cells and cells in the known disease state achieves an AUC of at least 0.95.

In various embodiments, the predictive model trained to distinguish between the morphological profiles of healthy cells and cells in the known disease state achieves an accuracy of at least 0.88. In various embodiments, the at least one cohort of synthetically pooled cells is built by randomly selecting a number of single cells or randomly selecting a number of tiles. In various embodiments, the synthetically pooled cells are formed by pooling together a plurality of cell lines of the known disease state or healthy state. In various embodiments, the plurality of cell lines are obtained from different subjects of the known disease state or healthy state. In various embodiments, pooling together the plurality of cell lines comprises combining embeddings or fixed feature vectors of randomly selected single cells. In various embodiments, combining the embeddings from the randomly selected single cells comprises averaging the embeddings or fixed feature vectors of the randomly selected single cells. In various embodiments, pooling together the plurality of cell lines does not involve physically pooling together the randomly selected single cells. In various embodiments, the at least one cohort of synthetically pooled cells are divided into separate training and testing folds for training the predictive model.

In various embodiments, the predictive model is trained by: capturing a plurality of images corresponding to the randomly selected single cells of the known disease state or healthy state; and using the plurality of images corresponding to the randomly selected single cells of the known disease state or healthy state to train the predictive model to distinguish between the morphological profiles of cells of the known disease state and cells of the healthy state. In various embodiments, using the plurality of images corresponding to the randomly selected single cells of the known disease state or healthy state to train the predictive model further comprises averaging embeddings of the plurality of images corresponding to the randomly selected single cells of the known disease state or healthy state. In various embodiments, the one or more cells of a common state comprise cells of a single cell line from a single subject. In various embodiments, analyzing the plurality of images for the one or more cells of a common state further comprises averaging embeddings from the one or more cells of a common state. In various embodiments, to distinguish between the morphological profiles of healthy cells and cells in the known disease state for the one or more cells of a common state, the predictive model is trained to compare an averaged embedding of the one or more cells of a common state to an averaged embedding of the plurality of images corresponding to the randomly selected single cells of the known disease state or healthy state.

In various embodiments, the predictive model is trained to predict the presence or absence of the known disease state with a prediction probability. In various embodiments, the healthy cells or the cells in the known disease state serve as a reference ground truth for training the predictive model. In various embodiments, the instructions when executed further cause the processor to: prior to capturing the plurality of images corresponding to the one or more cells of a common state, provide a perturbation to the one or more cells of a common state, the perturbation causing the one or more cells from a known disease state to an unknown disease state; subsequent to analyzing the plurality of images of the one or more cells of a common state, compare the predicted state of the one or more cells to the known disease state of the one or more cells known before providing the perturbation; and based on the comparison, identify the perturbation as having one of a therapeutic effect, a detrimental effect, or no effect. In various embodiments, the predictive model is one of a neural network, random forest, or regression model. In various embodiments, the neural network is a multilayer perceptron model. In various embodiments, the regression model is one of a logistic regression model or a ridge regression model.

In various embodiments, each of the morphological profiles comprises values of imaging features or comprises a transformed representation of images that define a known disease state or a healthy state of a cell. In various embodiments, the imaging features comprise one or more of cell features. In various embodiments, the cell features comprise one or more of cellular shape, cellular size, cellular organelles, object-neighbors features, mass features, intensity features, quality features, texture features, and global features. In various embodiments, the cell features are determined via fluorescently labeled biomarkers. In various embodiments, the cell features are determined via fluorescently labeled biomarkers identifying one or more of cell nucleus, cell nucleoli, plasma membrane, cytoplasmic RNA, endoplasmic reticulum, actin, Golgi apparatus, and mitochondria. In various embodiments, each cell in the one or more cells of a common state is one of a stem cell, a partially differentiated cell, or a terminally differentiated cell.

In various embodiments, each cell in the one or more cells of a common state is a somatic cell. In various embodiments, the somatic cell is a fibroblast or a peripheral blood mononuclear cell (PBMC). In various embodiments, the one or more cells of a common state are obtained from a subject through a tissue biopsy or blood draw. In various embodiments, the tissue biopsy is obtained from an extremity of the subject. In various embodiments, the morphological profile is extracted from a layer of a deep learning neural network. In various embodiments, the morphological profile is an averaged embedding representing a dimensionally reduced representation of values of the layer of the deep learning neural network. In various embodiments, the layer of the deep learning neural network is a penultimate layer of the deep learning neural network.

In various embodiments, the instructions when executed further cause the processor to: prior to capturing the plurality of images corresponding to the one or more cells of a common state, stain or have stained the one or more cells of a common state using one or more fluorescent dyes. In various embodiments, at least 5 cell features derive from fluorescently labeled biomarkers identifying plasma membrane. In various embodiments, at least 30 cell features derive from fluorescently labeled biomarkers identifying plasma membrane. In various embodiments, at least 5 cell features derive from fluorescently labeled biomarkers identifying cell nucleus. In various embodiments, at least 25 cell features derive from fluorescently labeled biomarkers identifying cell nucleus. In various embodiments, at least 5 cell features derive from fluorescently labeled biomarkers identifying endoplasmic reticulum. In various embodiments, at least 10 cell features derive from fluorescently labeled biomarkers identifying endoplasmic reticulum. In various embodiments, at least 5 cell features derive from fluorescently labeled biomarkers identifying mitochondria. In various embodiments, at least 35 cell features derive from fluorescently labeled biomarkers identifying mitochondria. In various embodiments, at least 5 cell features derive from fluorescently labeled biomarkers identifying RNA. In various embodiments, at least 10 cell features derive from fluorescently labeled biomarkers identifying RNA. In various embodiments, at least 60 correlated cell features derive from various fluorescence channels. In various embodiments, at least 20 correlated cell features derive from various fluorescence channels. In various embodiments, each of the plurality of images corresponding to the one or more cells of a common state corresponds to a fluorescent channel. In various embodiments, the steps of obtaining or having obtained the one or more cells of a common state and capturing the plurality of images corresponding to the one or more cells of a common state are performed in a high-throughput format using an automated array.

In various embodiments, the disease state of the cell predicted by the predictive model is a classification of at least two categories. In various embodiments, the at least two categories comprise a presence or absence of a neurodegenerative disease. In various embodiments, the at least two categories comprise a first subtype or a second subtype of a neurodegenerative disease. In various embodiments, the at least two categories further comprise a third subtype of the neurodegenerative disease. In various embodiments, the neurodegenerative disease is any one of Parkinson's Disease (PD), Alzheimer's Disease, Amyotrophic Lateral Sclerosis (ALS), Infantile Neuroaxonal Dystrophy (INAD), Multiple Sclerosis (MS), Amyotrophic Lateral Sclerosis (ALS), Batten Disease, Charcot-Marie-Tooth Disease (CMT), Autism, post-traumatic stress disorder (PTSD), schizophrenia, frontotemporal dementia (FTD), multiple system atrophy (MSA), and a synucleinopathy. In various embodiments, the first subtype comprises an LRRK2 subtype. In various embodiments, the second subtype comprises a sporadic PD subtype. In various embodiments, the third subtype comprises a GBA subtype.

In various embodiments, the instructions when executed further cause the processor to: identify a plurality of features associated with the known disease state when the one or more cells are predicted to be the known disease state; rank the plurality of features according to a degree of difference of the features between the known disease state and the healthy state; and select a list of top-ranked features according to a predefined threshold. In various embodiments, the instructions when executed further cause the processor to: filter the top-ranked features by removing a subset of features that are correlated; and update the list of top-ranked features by excluding the subset of features, where the updated list of top-ranked features are designated as a phenotype for characterizing the known disease state.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows a schematic disease prediction system for implementing a disease analysis pipeline, in accordance with an embodiment.

FIG. 2A is an example block diagram depicting the deployment of a predictive model, in accordance with an embodiment.

FIG. 2B is an example structure of a deep learning neural network for determining morphological profiles, in accordance with an embodiment.

FIG. 2C depicts an example process for creating synthetic pools for training a predictive mode, in accordance with an embodiment.

FIG. 3 is a flow process for training a predictive model for the disease analysis pipeline, in accordance with an embodiment.

FIG. 4 is a flow process for deploying a predictive model for the disease analysis pipeline, in accordance with an embodiment.

FIG. 5 is a flow process for identifying modifiers of disease state by deploying a predictive model, in accordance with an embodiment.

FIG. 6 depicts an example computing device for implementing system and methods described in reference to FIGS. 1-5.

FIGS. 7A-7D depict performance of a predictive model trained by using a synthetic pool and tested under different conditions.

FIGS. 8A-8D depict performance comparisons of predictive models trained with or without using a synthetic pool.

FIGS. 9A-9B show various summarizations of disease-specific features identified by a predictive model trained using a synthetic pool before and after correlation-related filtration.

### DETAILED DESCRIPTION

### Definitions

Terms used in the claims and specification are defined as set forth below unless otherwise specified.

As used in the specification and the appended claims, the singular forms “a,” “an” and “the” include plural referents unless the context clearly dictates otherwise.

The term “subject” encompasses a cell, tissue, or organism, human or non-human, whether male or female. In some embodiments, the term “subject” refers to a donor of a cell, such as a mammalian donor of more specifically a cell or a human donor of a cell.

The term “mammal” encompasses both humans and non-humans and includes but is not limited to humans, non-human primates, canines, felines, murines, bovines, equines, and porcines.

The phrase “morphological profile” refers to values of imaging features or a transformed representation of images that define a disease state of a cell. In various embodiments, a morphological profile of a cell includes cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features are extracted from images of cells that have been labeled using fluorescently labeled biomarkers. Other cell features include object-neighbors features, mass features, intensity features, quality features, texture features, and global features (e.g., cell counts, cell distances). In various embodiments, a morphological profile of a cell includes values of non-cell features such as information about a well that the cell resides within (e.g., well density, background versus signal, percent of touching cells in the well). In various embodiments, a morphological profile of a cell includes values of both cell features and non-cell features. In various embodiments, a morphological profile comprises a deep embedding vector extracted from a deep learning neural network that transforms values of images. For example, the morphological profile may be extracted from a penultimate layer of a deep learning neural network that analyzes images of cells.

The phrase “predictive model” refers to a machine-learned model that distinguishes between morphological profiles of cells of different disease states. Generally, a predictive model predicts the disease state of the cell based on the image features of a cell. In various embodiments, image features of the cell can be extracted from one or more images of the cell. In various embodiments, features of the cell can be structured as a deep embedding vector and are extracted from images via a deep learning neural network.

The phrase “obtaining a cell” encompasses obtaining a cell from a sample. The phrase also encompasses receiving a cell (e.g., from a third party).

The phrase “common state” refers to a feature(s) commonly shared by a number of cells. For example, based on the different features used in characterizing cells, a common state may refer to a common disease state, a common source, a common processing state, a common growth state, etc. Cells of a common disease state may indicate that the cells come from samples having a same disease or being in a healthy state. Cells of a common source may indicate that the cells come from samples collected from the same source such as the same institute, the same patient or same patient population, the same type of tissue or organ, etc. Cells of a common processing state may indicate that the cells come from samples that have been through the same processing procedure(s) such as the same cell isolation process, the same cell staining process, etc. Cells of a common growth state may indicate that the cells come from samples that share similar growth conditions. For example, the cells of a common growth state may indicate that these cells come from individuals having the same age range, or from samples having passed through a same period of growth in cell culture, etc.

The phrase “disease state” refers to a state of a cell. In various embodiments, the disease state refers to one of a presence or absence of a disease. For example, a disease state indicating absence of a disease may refer to a healthy state. In various embodiments, the disease state refers to a subtype of a disease. In particular embodiments, the disease is a neurodegenerative disease. For example, in the context of Parkinson's disease (PD), disease state refers to a presence or absence of PD. As another example, in the context of Parkinson's disease, the disease state refers to one of an LRRK2 subtype, a GBA subtype, or a sporadic subtype.

The phrase “phenotype” or “signature” refers to certain disease-specific features derived from images or their corresponding transformed representations from certain diseased cells.

The phrase “synthetic pool” refers to a pool of images or their transformed representations obtained from cells randomly selected from cell lines from different subjects (e.g., different donors) with a common disease state. In various embodiments, a synthetic pool may not require randomly selected cells to be physically pooled together. Instead, the cells in a synthetic pool used for imaging screens or other purposes may be from different wells and/or collected at different time points, as long as the cells in the synthetic pool originate from different cell lines from different donors with a common disease state. Therefore, a synthetic pool of cells can smooth out donor-specific features while highlighting disease specific features. In some embodiments, a synthetic pool of morphological profiles may be even dynamically updated by continuously adding morphological profiles when there are new donors that have a common disease state. In this context, a synthetic pool may be considered as a database or library that includes morphological profiles consistently updated for a disease state.

### Overview

In various embodiments, disclosed herein are methods and systems for performing high-throughput analysis of cells using a disease analysis pipeline that determines predicted disease states of cells by implementing a predictive model trained to distinguish between morphological profiles of cells of different disease states. Generally, the predictive model is trained using morphological profiles derived from a synthetic cohort of pooled cells. Here, a synthetically pooled cohort of cells represents cells pooled from different donors. This ensures that the morphological profiles derived from a synthetic cohort of pooled cells highlight disease-specific features while de-emphasizing donor-specific features, which are unlikely to be related to the disease. Altogether, by using synthetically pooled cohorts of cells during training of the predictive model, the predictive model can more effectively identify features that are indicative of the diseased state, while avoiding the confounding effects of the donor-specific features. Thus, predictive models trained using synthetically pooled cohorts of cells more accurately distinguish between morphological profiles of healthy cells and cells in the known disease state in comparison to a predictive model that is trained without using a cohort of synthetically pooled cells. In particular embodiments, the disease analysis pipeline determines predicted cellular disease states by implementing a predictive model trained to distinguish between morphological profiles of cells of the different disease states. Furthermore, a predictive model disclosed herein is useful for performing high-throughput drug screens, thereby enabling the identification of modifiers of disease states. Thus, modifiers of disease states identified using the predictive model can be implemented for therapeutic applications (e.g., by reverting a cell exhibiting a diseased state morphology towards a cell exhibiting a non-diseased state morphology). In particular embodiments, the disease analysis pipeline is useful for predicting neurodegenerative cellular disease states. In other embodiments, the disease analysis pipeline is useful for predicting cellular disease states for various diseases, examples of which are further described herein. Although the description herein may, at various points, refer to neurodegenerative diseases, the description herein may similarly be applied to various other diseases disclosed herein.

In various embodiments, the disease analysis pipeline disclosed herein further identifies certain features associated with a disease state to determine a presence or absence of the disease state. The disease-specific features may be considered as a phenotype of the disease state and may be determined based on a comparison of features of the disease state with features of non-disease states (e.g., healthy state or other different disease states). In particular embodiments, the disease analysis pipeline may use the morphological profiles of cells of known disease states to identify features associated with each disease state, so that the phenotype of each disease state can be then established. In particular embodiments, after establishing the phenotype of each disease state, the disease analysis pipeline may then focus on the phenotype of a disease state (while ignoring features not important for identification of the disease state) when determining the presence or absence of the disease state in the coming disease analysis.

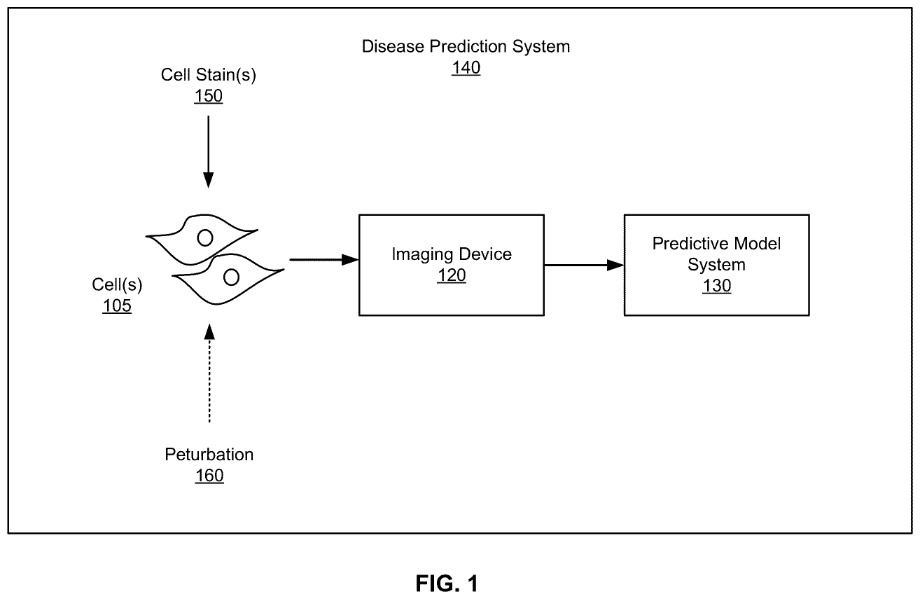

FIG. 1 shows an overall disease prediction system for implementing a disease analysis pipeline, in accordance with an embodiment. Generally, the disease prediction system 140 includes one or more cells 105 that are to be analyzed. In various embodiments, the one or more cells 105 are obtained from a single donor. In various embodiments, the one or more cells 105 are obtained from multiple donors. In various embodiments, the one or more cells 105 are obtained from at least 5 donors. In various embodiments, the one or more cells 105 are obtained from at least 10 donors, at least 20 donors, at least 30 donors, at least 40 donors, at least 50 donors, at least 75 donors, at least 100 donors, at least 200 donors, at least 300 donors, at least 400 donors, at least 500 donors, or at least 1000 donors.

In various embodiments, the cells 105 undergo a protocol for one or more cell stains 150. For example, cell stains 150 can be fluorescent stains for specific biomarkers of interest in the cells 105 (e.g., biomarkers of interest that can be informative for determining disease states of the cells 105). In various embodiments, the cells 105 can be exposed to a perturbation 160. Such a perturbation may have an effect on the disease state of the cell. In other embodiments, a perturbation 160 need not be applied to the cells 105, as indicated by the dotted line in FIG. 1.

The disease prediction system 140 includes an imaging device 120 that captures one or more images of the cells 105. The predictive model system 130 analyzes the one or more captured images of the cells 105. In various embodiments, the predictive model system 130 analyzes one or more captured images of multiple cells 105 and predicts the disease states of the multiple cells 105. In various embodiments, the predictive model system 130 analyzes one or more captured images of a single cell to predict the disease state of the single cell. For example, the predictive model system 130 may analyze features associated with the phenotype of a disease state to determine a presence or absence of the disease state.

In various embodiments, the predictive model system 130 analyzes one or more captured images of the cells 105, where different images are captured using different imaging channels. Therefore, different images include signal intensity indicating presence/absence of cell stains 150. Thus, the predictive model system 130 determines and selects cell stains that are informative for predicting the disease state of the cells 105.

In various embodiments, the predictive model system 130 analyzes one or more captured images of the cells 105, where the cells 105 have been exposed to a perturbation 160. Thus, the predictive model system 130 can determine the effects imparted by the perturbation 160. As one example, the predictive model system 130 can analyze a first set of images of cells captured before exposure to a perturbation 160 and a second set of images of the same cells captured after exposure to the perturbation 160. Thus, the change in the disease state prior to and subsequent to exposure to the perturbation 160 can represent the effects of the perturbation 160. For example, a cell (or a number of cells from a cell line) may exhibit a disease state prior to exposure to the perturbation. If subsequent to exposure, the cell(s) exhibit a morphological profile (or averaged morphological profile from a number of cells) that is more similar to a non-diseased state, the perturbation 160 can be characterized as having a therapeutic effect that reverts the cell(s) towards a healthier morphological profile and away from a diseased morphological profile.

Altogether, the disease prediction system 140 prepares cells 105 (e.g., exposes cells 105 to cell stains 150 and/or perturbation 160), captures images of the cells 105 using the imaging device 120, and predicts disease states of the cells 105 using the predictive model system 130. In various embodiments, the disease prediction system 140 is a high-throughput system that processes cells 105 in a high-throughput manner such that large populations of cells are rapidly prepared and analyzed to predict cellular disease states. The imaging device 120 may, through automated means, prepare cells (e.g., seed, culture, and/or treat cells), capture images from the cells 105, and provide the captured images to the predictive model system 130 for analysis. Additional descriptions regarding the automated hardware and processes for handling cells are described herein. Further descriptions regarding automated hardware and processes for handling cells are described in Paull, D., et al. Automated, high-throughput derivation, characterization and differentiation of induced pluripotent stem cells. Nat Methods 12, 885-892 (2015), which is incorporated by reference in its entirety.

### Predictive Model System

Generally, the predictive model system (e.g., predictive model system 130 described in FIG. 1) analyzes one or more images including cells that are captured by the imaging device 120. In various embodiments, the predictive model system analyzes images of cells for training a predictive model. In various embodiments, the predictive model system analyzes images of cells for deploying a predictive model to predict disease states of a cell in the images. In various embodiments, the predictive model system and/or predictive models analyze captured images by at least analyzing values of features of the images (e.g., by extracting values of the features from the images or by deploying a neural network that extracts features from the images in the form of a deep embedding vector).

In particular embodiments, the predictive model system analyzes images from a synthetic pool and uses averaged features extracted from the images of the synthetic pool to train the predictive model. In various embodiments, the predictive model system further identifies features associated with a specific disease state, and generates a phenotype for the disease state based on the identified features specific to the disease state.

In various embodiments, the images include fluorescent intensities of dyes that were previously used to stain certain components or aspects of the cells. In various embodiments, the images may have undergone Cell Paint staining and therefore, the images include fluorescent intensities of Cell Paint dyes that label cellular components (e.g., one or more of cell nucleus, cell nucleoli, plasma membrane, cytoplasmic RNA, endoplasmic reticulum, actin, Golgi apparatus, and mitochondria). Cell Paint is described in further detail in Bray et al., Cell Painting, a high-content image-based assay for morphological profiling using multiplexed fluorescent dyes. Nat. Protoc. 2016 September; 11(9): 1757-1774 as well as Schiff, L. et al., Deep Learning and automated Cell Painting reveal Parkinson's disease-specific signatures in primary patient fibroblasts, bioRxiv 2020.11.13.380576, each of which is hereby incorporated by reference in its entirety. In various embodiments, each image corresponds to a particular fluorescent channel (e.g., a fluorescent channel corresponding to a range of wavelengths). Therefore, each image can include fluorescent intensities arising from a single fluorescent dye with limited effect from other fluorescent dyes.

In various embodiments, prior to feeding the images to the predictive model (e.g., either for training the predictive model or for deploying the predictive model), the predictive model system performs image processing steps on the one or more images. Generally, the image processing steps are useful for ensuring that the predictive model can appropriately analyze the processed images. As one example, the predictive model system can perform a correction or a normalization over one or more images. For example, the predictive model system can perform a correction or normalization across one or more images to ensure that the images are comparable to one another. This ensures that extraneous factors do not negatively impact the training or deployment of the predictive model. An example correction can be a flatfield image correction. Another example correction can be an illumination correction which corrects for heterogeneities in the images that may arise from biases arising from the imaging device 120. Further description of illumination correction in Cell Paint images is described in Bray et al., Cell Painting, a high-content image-based assay for morphological profiling using multiplexed fluorescent dyes. Nat. Protoc. 2016 September; 11(9): 1757-1774, which is hereby incorporated by reference in its entirety.

In various embodiments, the image processing steps involve performing image segmentation. For example, if an image includes multiple cells, the predictive model system performs an image segmentation such that the resulting images each include a single cell. For example, if a raw image includes Y cells, the predictive model system may segment the image into Y different processed images, where each resulting image includes a single cell. In various embodiments, the predictive model system implements a nuclei segmentation algorithm to segment the images. Thus, a predictive model can subsequently analyze the processed images on a per-cell basis.

Generally, in analyzing one or more images, the predictive model analyzes values of features of the images. In various embodiments, the predictive model analyzes image features which can be extracted from the one or more images. For example, such image features can be extracted from the one or more images using a feature extraction algorithm. Image features can include: cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features can be extracted from images of cells that have been labeled using fluorescently labeled biomarkers. Other cell features include colocalization features, radial distribution features, granularity features, object-neighbors features, mass features, intensity features, quality features, texture features, and global features. In various embodiments, image features include non-cell features such as information about a well that the cell resides within (e.g., well density, background versus signal, percent of touching cells in the well). In various embodiments, image features include CellProfiler features, examples of which are described in further detail in Carpenter, A. E., et al. CellProfiler: image analysis software for identifying and quantifying cell phenotypes. Genome Biol 7, R100 (2006), which is incorporated by reference in its entirety. In various embodiments, the values of features of the images are a part of a morphological profile of the cell. In various embodiments, to determine a predicted disease state of the cell, the predictive model compares the morphological profile of the cell (e.g., values of features of the images) extracted from an image to values of features for morphological profiles of other cells of known disease state (e.g., other cells of known disease state that were used during training of the predictive model). For example, the predictive model compares the morphological profile of the cell (e.g., values of features of the images) extracted from an image to averaged values of features for morphological profiles of other cells from multiple donors of the known disease state. Further description of morphological profiles of cells and averaged values of features for the morphological profiles of other cells from multiple donors is provided herein.

In various embodiments, a neural network is employed that analyzes the images and extracts relevant feature values. For example, the neural network receives the images as input and identifies relevant features. In various embodiments, the relevant features identified by the neural network represent non-interpretable features that represent sophisticated features that are not readily interpretable. In such embodiments, the features identified by the neural network can be structured as a deep embedding vector, which is a transformed representation of the images. Values of these features identified by the neural network can be provided to the predictive model for analysis. In one example, the analysis may include generating average values for each of these features based on the features identified by the neural network from multiple cell lines from different donors.

In various embodiments, a morphological profile is composed of at least 2 features, at least 3 features, at least 4 features, at least 5 features, at least 10 features, at least 20 features, at least 30 features, at least 40 features, at least 50 features, at least 75 features, at least 100 features, at least 200 features, at least 300 features, at least 400 features, at least 500 features, at least 600 features, at least 700 features, at least 800 features, at least 900 features, at least 1000 features, at least 1100 features, at least 1200 features, at least 1300 features, at least 1400 features, or at least 1500 features. In particular embodiments, a morphological profile is composed of at least 1000 features. In particular embodiments, a morphological profile is composed of at least 1100 features. In particular embodiments, a morphological profile is composed of at least 1200 features. In particular embodiments, a morphological profile is composed of 1200 features.

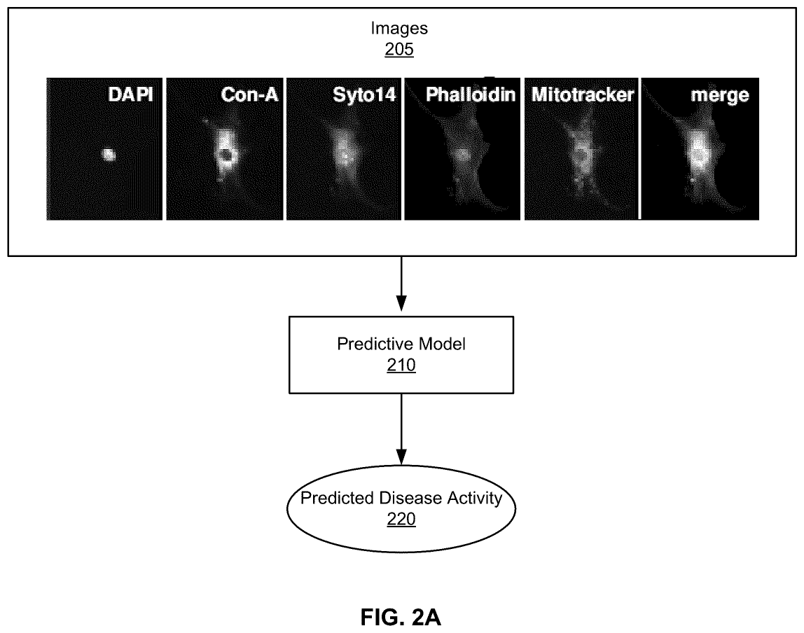

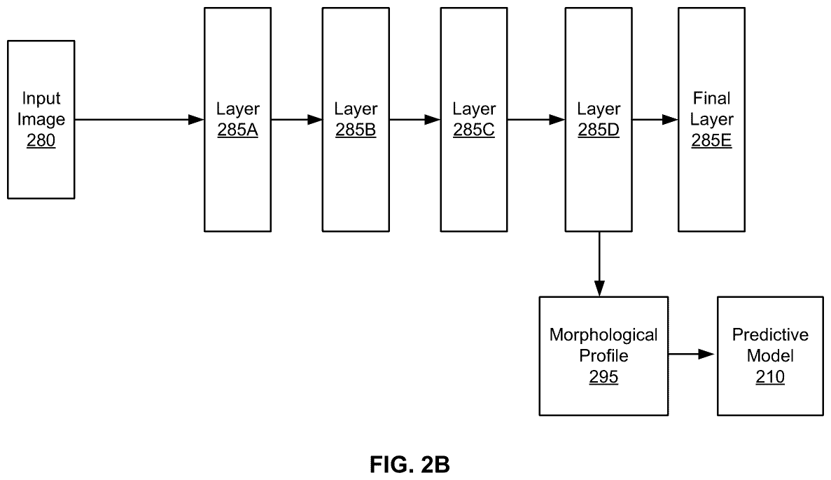

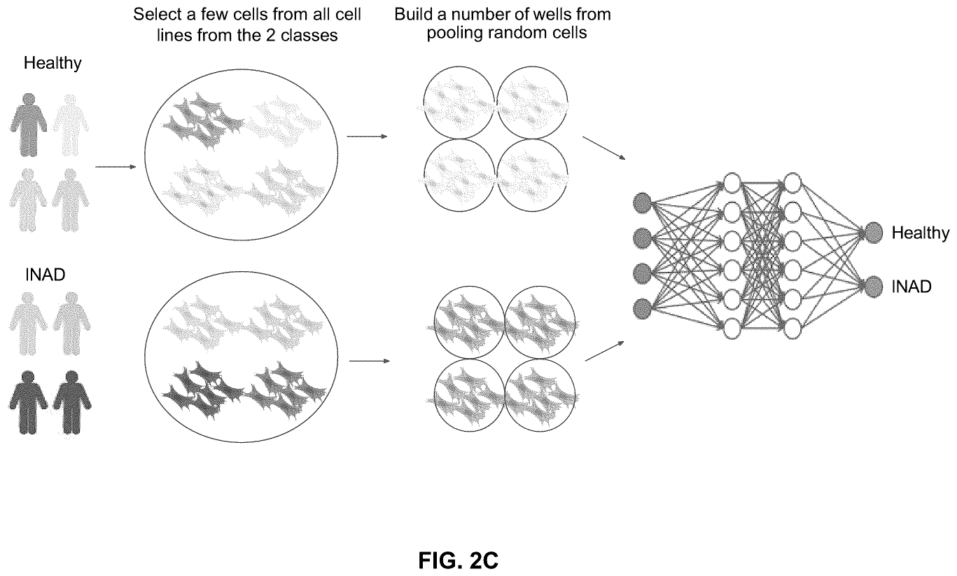

In various embodiments, the predictive model analyzes multiple images or features of the multiple images of a cell across different channels that have fluorescent intensities for different fluorescent dyes. Reference is now made to FIG. 2A, which is a block diagram that depicts the deployment of the predictive model, in accordance with an embodiment. FIG. 2A shows the multiple images 205 of a single cell. Here, each image 205 corresponds to a particular channel (e.g., fluorescent channel) which depicts fluorescent intensity for a fluorescent dye that has stained a marker of the cell. For example, as shown in FIG. 2A, a first image includes fluorescent intensity from a DAPI stain which shows the cell nucleus. A second image includes fluorescent intensity from a concanavalin A (Con-A) stain which shows the cell surface. A third image includes fluorescent intensity from a Syto14 stain which shows nucleic acids of the cell. A fourth image includes fluorescent intensity from a Phalloidin stain which shows actin filament of the cell. A fifth image includes fluorescent intensity from a Mitotracker stain which shows mitochondria of the cell. A sixth image includes the merged fluorescent intensities across the other images. Although FIG. 2A depicts six images with particular fluorescent dyes (e.g., images 205), in various embodiments, additional or fewer images with same or different fluorescent dyes may be employed. For example, additional or alternative stains can include any of Alexa Fluor® 488 Conjugate (Invitrogen™ C11252), Alexa Fluor® 568 Phalloidin (Invitrogen™ A12380), Hoechst 33342 trihydrochloride, trihydrate (Invitrogen™ H3570), Molecular Probes Wheat Germ Agglutinin, or Alexa Fluor 555 Conjugate (Invitrogen™ W32464).

As shown in FIG. 2A, the multiple images 205 from a cell can be provided as input to a predictive model 210. In various embodiments, a feature extraction process is performed on the multiple images 205 and the values of the extracted features for the cell are provided as input to the predictive model 210. In various embodiments, a feature extraction process involves implementing a deep learning neural network to generate deep embeddings that can be provided as input to the predictive model 210. The predictive model 210 determines a predicted disease state 220 for the cell in the images 205. The process can be repeated for other sets of images corresponding to other cells such that the predictive model 210 analyzes each other set of images to predict the disease states of each of the other cells. In various embodiments, images from multiple cells from a single donor or a single cell line are collected, and a process can be performed for the multiple cells by averaging the extracted features or embeddings from the multiple cells, which then is input into the prediction model 210 to predict the disease state of the multiple cells like a pool. In various embodiments, the predictive model 210 predicts a disease state of a disease described herein. In various embodiments, the predictive model 210 predicts a disease state of a neurodegenerative disease. In particular embodiments, the neurodegenerative disease is Parkinson's disease (PD). Thus, the predictive model 210 may predict a presence or absence of PD. As another example, the predictive model 210 may predict a presence of a subtype of PD, such as an LRRK2 subtype, a GBA subtype, or a sporadic subtype. In other embodiments, the neurodegenerative disease is Infantile Neuroaxonal Dystrophy (INAD). Thus the predictive model 210 may predict a presence or absence of INAD for a single cell or multiple cells as a group if these cells originate from a single donor or a single cell line.

In various embodiments, the predicted disease state 220 of the cell(s) can be compared to a previous disease state of the cell(s). For example, the cell(s) may have previously undergone a perturbation (e.g., by exposure to a drug), which may have had an effect on the disease state of the cell(s). Prior to the perturbation, the cell(s) may have a previous disease state. Thus, the previous disease state of the cell(s) is compared to the predicted disease state 220 to determine the effects of the perturbation. This is useful for identifying perturbations that are modifiers of cellular disease state.

### Predictive Model

Generally, the predictive model analyzes a morphological profile (e.g., features extracted from an image with one or more cells) of the one or more cells and outputs a prediction of the disease state of the one or more cells in the image. In various embodiments, the predictive model can be any one of a regression model (e.g., linear regression, logistic regression, or polynomial regression), decision tree, random forest, support vector machine, Naïve Bayes model, k-means cluster, or neural network (e.g., feed-forward networks, multilayer perceptron networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks). In various embodiments, the predictive model comprises a dimensionality reduction component for visualizing data, the dimensionality reduction component comprising any of a principal component analysis (PCA) component or a T-distributed Stochastic Neighbor Embedding (TSNe). In particular embodiments, the predictive model is a neural network. In particular embodiments, the predictive model is a random forest. In particular embodiments, the predictive model is a regression model.

In various embodiments, the predictive model includes one or more parameters, such as hyperparameters and/or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, variables and threshold for splitting nodes in a random forest, support vectors in a support vector machine, and coefficients in a regression model. The model parameters of the predictive model are trained (e.g., adjusted) using the training data to improve the predictive power of the predictive model.

In various embodiments, the predictive model outputs a classification of a disease state of a cell or a group of cells. In various embodiments, the predictive model outputs one of two possible classifications of a disease state of a cell. For example, the predictive model classifies the cell(s) as either having a presence of a disease or absence of a disease (e.g., neurodegenerative disease). As another example, the predictive model classifies the cell(s) in one of multiple possible subtypes of a disease (e.g., neurodegenerative disease). For example, the predictive model may classify the cell(s) in one of at least 2, at least 3, at least 4, at least 5, at least 6, at least 7, at least 8, at least 9, or at least 10 different subtypes. In particular embodiments, the predictive model classifies the cell(s) in one of two possible subtypes of a disease. For example, in the context of Parkinson's Disease, the predictive model may classify the cell(s) in one of either an LRRK2 subtype or a sporadic PD subtype.

In various embodiments, the predictive model outputs one of three possible classifications of a disease state of a cell or a group of cells. For example, the predictive model classifies the cell(s) in one of three possible subtypes of a disease (e.g., neurodegenerative disease). In the context of Parkinson's Disease, the predictive model may classify the cell(s) in one of any of an LRRK2 subtype, a GBA subtype, or a sporadic PD subtype.

The predictive model can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, gradient descent, and dimensionality reduction techniques such as manifold learning, principal component analysis, factor analysis, autoencoder regularization, and independent component analysis, or combinations thereof. In particular embodiments, the predictive model is trained using a deep learning algorithm. In particular embodiments, the predictive model is trained using a random forest algorithm. In particular embodiments, the predictive model is trained using a linear regression algorithm. In various embodiments, the predictive model is trained using supervised learning algorithms, unsupervised learning algorithms, semi-supervised learning algorithms (e.g., partial supervision), weak supervision, transfer, multi-task learning, or any combination thereof. In particular embodiments, the predictive model is trained using a weak supervision learning algorithm.

In various embodiments, the predictive model is trained to improve its ability to predict the disease state of a cell or a group of cells using training data that include reference ground truth values. For example, a reference can be a known disease state of a cell or a group of cells. In a training iteration, the predictive model analyzes images acquired from the cell(s) and determines a predicted disease state of the cell(s). The predicted disease state of the cell(s) can be compared against the reference ground truth value (e.g., known disease state of the cell(s)) and the predictive model is tuned to improve the prediction accuracy. For example, the parameters of the predictive model are adjusted such that the predictive model's prediction of the disease state of the cell is improved. In particular embodiments, the predictive model is a neural network and therefore, the weights associated with nodes in one or more layers of the neural network are adjusted to improve the accuracy of the predictive model's predictions. In various embodiments, the parameters of the neural network are trained using backpropagation to minimize a loss function. Altogether, over numerous training iterations across different cells or different groups of cells, the predictive model is trained to improve its prediction of cellular disease states across the different cells or different groups of cells.

In various embodiments, the predictive model is trained on features of images acquired from cells of known disease state. Here, features may be imaging features such as cell features and/or non-cell features. In various embodiments, features may be organized as a deep embedding vector. For example, a deep neural network can be employed that analyzes images to determine a deep embedding vector (e.g., a morphological profile) of a cell. For another example, if a group of cells (e.g., cells randomly selected from different cell lines from different donors) are used in a single training iteration, the deep neural network can be employed that analyzes images from each cell in the synthetic pool to determine a deep embedding vector of each cell and then combine the deep embedding vectors of the group of cells to determine a combined deep embedding vector representing the group of cells. An example of such a deep neural network is described above in reference to FIG. 2B. Here, at each training iteration, the predictive model is trained to predict the disease state using the deep embedding vector (e.g., a morphological profile) from a single cell or a combined deep embedding vector from a group of cells in a synthetic pool. In some embodiments, by using the averaged deep embedding vector of the synthetic pool, the donor-specific variation that may hide the features characterizing a disease state can be avoided. An example illustration of using a synthetic pool to train the predictive model is further described below in reference to FIG. 2C.

In FIG. 2C, a process for training a predictive model using a synthetic pool is illustrated by taking INAD as an example disease. As illustrated, a group of donors from a known disease state (e.g., from patients known to have INAD disease) can be recruited to collect cell lines from these donors. Cells from each cell line from each donor can be then randomly selected. Images of randomly selected cells can be then extracted, e.g., by using a deep neural network, for establishing the morphological profile (e.g., deep embedding vector) for each randomly selected cell. In some embodiments, a morphological profile is comprised of fixed feature vectors extracted from each randomly selected cell. After obtaining the morphological profile of each randomly selected cell, the morphological profiles of these randomly selected cells are then combined to obtain a combined morphological profile representing the randomly selected cells.

Generally, the step of combining morphological profiles of randomly selected cells represents the step of synthetic pooling. Thus, the synthetic pooling does not involve physical pooling of randomly selected cells, but instead, involves in silico combining of morphological profiles of randomly selected cells. In various embodiments, combining morphological profiles of different cells comprises determining a statistical combination of morphological profiles of different cells. Example statistical combinations include an average, a median, a mode, a maximum value, a minimum value, a summation, a variance, or a standard deviation. In particular embodiments, combining morphological profiles of different cells comprises determining an average of morphological profiles of different cells.

In various embodiments, a large number of combined morphological profiles can be similarly obtained, which can be used as a dataset for training the predictive model. In various embodiments, combined morphological profiles include a combination of morphological profiles of at least 2 cells, at least 3 cells, at least 4 cells, at least 5 cells, at least 6 cells, at least 7 cells, at least 8 cells, at least 9 cells, at least 10 cells, at least 11 cells, at least 12 cells, at least 13 cells, at least 14 cells, at least 15 cells, at least 16 cells, at least 17 cells, at least 18 cells, at least 19 cells, at least 20 cells, at least 25 cells, at least 30 cells, at least 35 cells, at least 40 cells, at least 45 cells, at least 50 cells, at least 60 cells, at least 70 cells, at least 80 cells, at least 90 cells, at least 100 cells, at least 200 cells, at least 300 cells, at least 400 cells, at least 500 cells, at least 600 cells, at least 700 cells, at least 800 cells, at least 900 cells, at least 1000 cells, at least 2000 cells, at least 3000 cells, at least 4000 cells, at least 5000 cells, at least 6000 cells, at least 7000 cells, at least 8000 cells, at least 9000 cells, at least 10000 cells, at least 20000 cells, at least 30000 cells, at least 40000 cells, at least 50000 cells, at least 60000 cells, at least 70000 cells, at least 80000 cells, at least 90000 cells, or at least 100000 cells.

In various embodiments, the dataset can be divided into three folds, with two folds being used for training the predictive model and the held-out fold being used for testing. The as-trained predictive model can be used to predict a presence or absence of the disease (e.g., INAD) at a cell level or at a well level by averaging the morphological profiles of randomly selected cells from a well. In various embodiments, predictive models for any disease state can be trained in this way by using a synthetic pool. For example, for the PD disease that contains three different subtypes, each subtype can be trained in this way to allow the predictive model to predict the presence or absence of each specific subtype.

Referring back to FIG. 2B, in various embodiments, a trained predictive model includes a plurality of morphological profiles (which can be a plurality of combined morphological profiles) that each defines cells of different disease states. In various embodiments, a morphological profile for a cell of a particular disease state refers to a combination of values of features that define the cell of the particular disease state. For example, a morphological profile for a cell of a particular disease state may be a feature vector including values of features that are informative for defining the cell of the particular disease state. Thus, a second morphological profile for a cell of a different disease state can be a second feature vector including different values of the features that are informative for defining the cell of the different disease state. With respect to synthetic pools, a combined morphological profile for a synthetic pool of a particular state may be a combined feature vector including combined values of features that are informative for defining the cells of the synthetic pool in the particular disease state. In addition, a second combined morphological profile (including combined values of features) for a synthetic pool of cells of a second disease state can be different from a first combined morphological profile (including combined values of features) for another synthetic pool of cells of a first disease state.

In various embodiments, a morphological profile of a cell includes image features that are extracted from one or more images of the cell. Image features can include cell features (e.g., cell morphological features) including cellular shape and size as well as cell characteristics such as organelles including cell nucleus, cell nucleoli, plasma membrane, cytoplasmic RNA, endoplasmic reticulum, actin, Golgi apparatus, and mitochondria. In various embodiments, values of cell features can be extracted from images of cells that have been labeled using fluorescently labeled biomarkers. Other cell features include object-neighbors features, mass features, intensity features, quality features, texture features, and global features. In various embodiments, image features include non-cell features such as information about a well that the cell resides within (e.g., well density, background versus signal, percent of touching cells in the well). In various embodiments, each image feature, either cell feature or non-cell feature, from multiple cells can be averaged to get an averaged feature to represent the image feature of the multiple cells.

In various embodiments, a morphological profile for a cell can include non-interpretable features that are determined using a neural network. Here, the morphological profile can be a representation of the images from which the non-interpretable features were derived. In various embodiments, in addition to non-interpretable features, the morphological profile can also include imaging features (e.g., cell features or non-cell features). For example, the morphological profile may be a vector including both non-interpretable features and image features. In various embodiments, the morphological profile may be a vector including CellProfiler features.

In various embodiments, a morphological profile for a cell can be developed using a deep learning neural network comprised of multiple layers of nodes. The morphological profile can be an embedding derived from a layer of the deep learning neural network that is a transformed representation of the images. In various embodiments, the morphological profile is extracted from a layer of the neural network. As one example, the morphological profile for a cell can be extracted from the penultimate layer of the neural network. As one example, the morphological profile for a cell can be extracted from the third to last layer of the neural network. In this context, the transformed representation refers to values of the images that have at least undergone transformations through the preceding layers of the neural network. Thus, the morphological profile can be a transformed representation of one or more images. In various embodiments, an embedding is a dimensionally reduced representation of values in a layer. Thus, an embedding can be used comparatively by calculating the Euclidean distance between the embedding and other embeddings of cells of known disease states as a measure of phenotypic distance.

In various embodiments, the morphological profile is a deep embedding vector with X elements. In various embodiments, the deep embedding vector includes 64 elements. In various embodiments, the morphological profile is a deep embedding vector concatenated across multiple vectors to yield X elements. For example, given 5 image channels (e.g., image channels of DAPI, Con-A, Syto14, Phalloidin, and Mitotracker), the deep embedding vector can be a concatenation of vectors from the 5 image channels. Given 64 elements for each image channel, the deep embedding vector can be a 320-dimensional vector representing the concatenation of the 5 separate 64 element vectors.

Reference is now made to FIG. 2B, which depicts an example structure of a deep learning neural network 275 for determining morphological profiles, in accordance with an embodiment. Here, the input image 280 is provided as input to a first layer 285A of the neural network. For example, the input image 280 can be structured as an input vector and provided to nodes of the first layer 285A. The first layer 285A transforms the input values and propagates the values through the subsequent layers 285B, 285C, and 285D. The deep learning neural network 275 may terminate in a final layer 285E. In various embodiments, the layer 285D can represent the morphological profile 295 of the cell and can be a transformed representation of the input image 280. In this scenario, the morphological profile 295 can be composed of non-interpretable features that include sophisticated features determined by the neural network.

As shown in FIG. 2B, the morphological profile 295 can be provided to the predictive model 210. In various embodiments, the predictive model 210 may compare the morphological profile 295 of the cell to morphological profiles of cells of known disease states. For example, if the morphological profile 295 of the cell is similar to a morphological profile of a cell of a known disease state, then the predictive model 210 can predict that the state of the cell is also of the known disease state.

Put more generally, in predicting the disease state of a cell, the predictive model can compare the values of features of the cell (or a transformed representation of images of the cell) to values of features (or a transformed representation of images of the cell) of one or more morphological profiles of cells of known disease state. For example, if the values of features (or transformed representation of images of the cell) of the cell are closer to values of features (or transformed representation of images) of a first morphological profile in comparison to values of features (or a transformed representation of images) of a second morphological profile, the predictive model can predict that the disease state of the cell is the disease state corresponding to the first morphological profile.

In various embodiments, morphological profile 295 is obtained from each of a plurality of cells (e.g., cells randomly selected from a well of cells from a single cell line and/or a single donor). The obtained multiple morphological profiles from the randomly selected cells are then combined to obtain a combined morphological profile. The combined morphological profile is then input into the predictive model 210. The predictive model 201 then compares the combined morphological profile, representing the randomly selected cells, with the morphological profiles of cells of known disease states, to determine a presence or absence of a specific disease. For example, in the case of the PD disease, the predictive model 201 may compare the combined morphological profile with morphological profiles of each PD subtype and with the morphological profile of healthy cells, to determine the disease state (e.g., a specific PD subtype or healthy state) of the randomly selected cells form a single cell line and/or single donor.

In various embodiments, the predictive model may include additional functions besides the above-described prediction of disease states of cells. In particular embodiments, the predictive model may determine specific features associated with a disease state. For example, after determining the morphological profiles of cells associated with various disease states, the predictive model may compare the morphological profiles of the various disease states, and determine certain features that are specific to a disease state. This may include comparing the morphological profiles of cells of a known disease state with morphological profiles of cells of healthy state and/or other disease states, and then determining which features included in the morphological profile are specific to a known disease state but not to healthy state or other disease states. In various embodiments, a threshold may be established for each feature to determine whether a difference is considered significant.

In various embodiments, to prevent donor-specific variations from affecting the identification of features characterizing the disease state, the predictive model may use the combined morphological profile established from a synthetic pool with a known disease state in the comparison process. That is, the combined morphological profile from a synthetic pool of a known disease state is compared to the combined morphological profiles of other disease states (e.g., healthy state or other subtypes of a disease) to determine features specific to the known disease state.

In various embodiments, when determining whether a feature is specific to a disease state, the features detectable from Cell Paint stains may be ranked according to their specificity to the disease state, for example, according to a difference of a feature value between the disease state and non-disease state or according to other possible means. Accordingly, for every feature that shows a difference, these features may be ranked according to the significance of difference, to generate a feature ranking list specific to the disease state. The more obvious difference, the higher the rank.

In various embodiments, after the features specific to a disease state is determined and ranked, certain features that are correlated may be removed from the ranking list since these features may always relate to each other, and thus detection of one feature is normally enough to tell the other correlated features. To save time and cost in imaging and later processing, some of the correlated features can be removed from the ranking list. For example, if there are three features that are always correlated, and detection of one feature can tell the remaining other two features, then only one of the three features remains in the ranking list. In particular embodiments, the ranking list for a specific disease state may include top 10, top 15, top 20, top 30, top 40, etc.

In various embodiments, by determining the top-ranked features specific to the known disease state, the features may allow establishing a phenotype for identifying the disease state (e.g., for predicting the disease state using the predictive model). For example, after determining the features specific to a disease state, the disease state prediction process for determining a presence or absence of the disease state may be focused on these top-ranked features specific to the disease state, but ignore features ranked low or non-specific to the disease state. This includes using stains specific to the determined top-ranked features and/or processing images by focusing on these features. In some embodiments, the exact number of top-ranked features selected for disease state prediction may vary for each specific disease state and may depend on the capacities of the imaging device and predictive model, among others.

### Methods for Determining Cellular Disease State

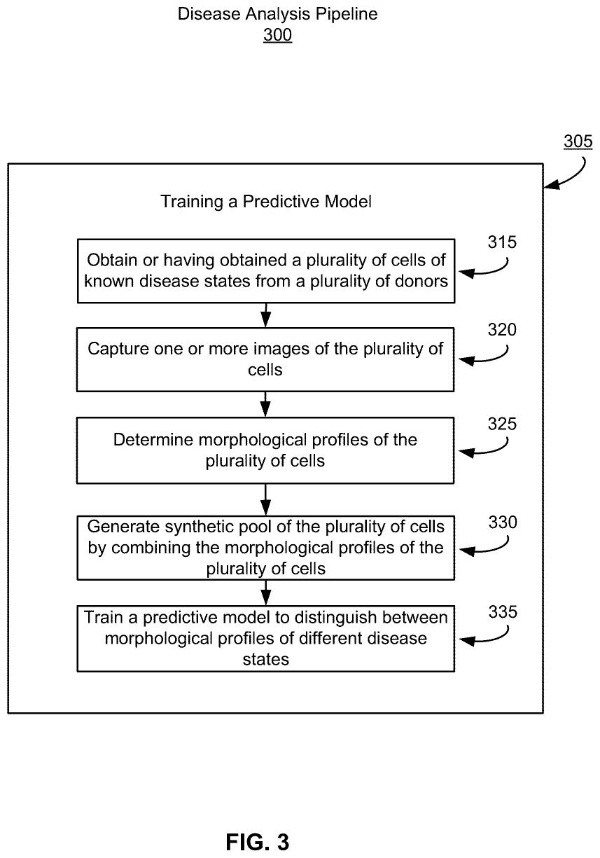

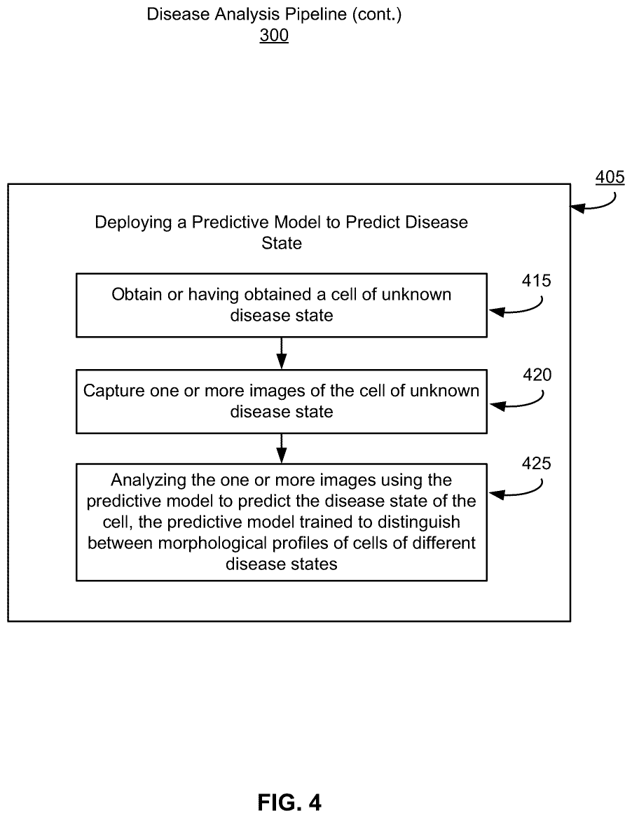

Methods disclosed herein describe the disease analysis pipeline. FIG. 3 is a flow process for training a predictive model for the disease analysis pipeline, in accordance with an embodiment. Furthermore, FIG. 4 is a flow process for deploying a predictive model for the disease analysis pipeline, in accordance with an embodiment.

Generally, the disease analysis pipeline 300 refers to the deployment of a predictive model for predicting the disease state of a cell, as is shown in FIG. 4. In various embodiments, the disease analysis pipeline 300 further refers to the training of a predictive model as is shown in FIG. 3. Thus, although the description below may refer to the disease analysis pipeline as incorporating both the training and deployment of the predictive model, in various embodiments, the disease analysis pipeline 300 only refers to the deployment of a previously trained predictive model.

Referring first to FIG. 3, at step 305, the predictive model is trained. Here, the training of the predictive model includes steps 315, 320, 325, 330, and 335. Step 315 involves obtaining or having obtained a plurality of cells of known disease states from a plurality of donors. For example, the plurality of cells may have been obtained from a number of donors of known disease states (e.g., from INAD patients or healthy donors). The plurality of cells may have been randomly selected from the plurality of donors. Step 320 involves capturing one or more images for the plurality of cells. As an example, the plurality of cells may have been stained (e.g., with Cell Paint stains) and therefore, the different images of each of the plurality of cells correspond to different fluorescent channels that include fluorescent intensity indicating the cell nuclei, nucleic acids, endoplasmic reticulum, actin/Golgi/plasma membrane, and mitochondria, etc.

Step 325 involves determining the morphological profiles of the plurality of cells. In various embodiments, a feature extraction process can be performed on the one or more images of the plurality of randomly selected cells. Thus, extracted features can be included in the morphological profile of each randomly selected cell. As another example, the morphological profile may comprise a transformed representation of the one or more images for the randomly selected cell. Here, the morphological profile may be a deep embedding vector that includes non-interpretable features derived by a neural network.

Step 330 involves generating a synthetic pool of the plurality of cells by combining the morphological profiles of the plurality of cells. For example, after obtaining the morphological profile of each randomly selected cell, the morphological profiles of these randomly selected cells are then pooled together and combined to get obtain a combined morphological profile representing the randomly selected cells of a known disease state. In various embodiments, the generation of the synthetic pool does not involve physical pooling of the randomly selected cells, but instead, involves in silico combining of morphological profiles of randomly selected cells. In various embodiments, combining morphological profiles of different cells comprises determining a statistical combination of morphological profiles of different cells.

Step 335 involves training a predictive model to distinguish between morphological profiles of cells of different disease states using combined morphological profiles. In various embodiments, the predictive model learns combined morphological profiles of cells of different diseased states. For example, the combined morphological profiles may include extracted and combined imaging features that enable the predictive model to differentiate combined morphological profiles of cells between different diseased states. Given the reference ground truth values (e.g., a known disease state) for the randomly selected cells, the predictive model is trained to improve its prediction of the disease states of the randomly selected cells. For example, as the combined morphological profiles have minimized the effects caused by donor-specific variations, the predictive model is trained to improve its prediction by identifying features that are more obvious in characterizing the known disease state when compared to the morphological profiles that are not combined.

Referring now to FIG. 4, at step 405, a trained predictive model is deployed to predict the cellular disease state of a cell. Here, the deployment of the predictive model includes steps 415, 420, and 425. Step 415 involves obtaining or having obtained a cell or a number of cells of an unknown disease state. As one example, the cell(s) may be derived from a subject and therefore, is evaluated for the disease state for purposes of diagnosing the subject with a disease. As another example, the cell(s) may have been perturbed (e.g., perturbed using a small molecule drug), and therefore, the perturbation caused the cell(s) to alter its morphological behavior corresponding to a different disease state. Thus, the predictive model is deployed to determine whether the disease state of the cell(s) has changed due to the perturbation.

Step 420 involves capturing one or more images of the cell(s) of unknown disease state. As an example, the cell may have been stained (e.g., with Cell Paint stains) and therefore, the different images of the cell(s) correspond to different fluorescent channels that include fluorescent intensity indicating the cell nuclei, nucleic acids, endoplasmic reticulum, actin/Golgi/plasma membrane, and mitochondria.

Step 425 involves analyzing the one or more images using the predictive model to predict the disease state of the cell. Here, the predictive model was previously trained to distinguish between morphological profiles of cells of different disease states. Thus, in some embodiments, the predictive model predicts a disease state of the cell(s) by comparing the morphological profile of the cell, or the averaged morphological profile of the number of cells from the subject, with morphological profiles of cells of known disease states.

### Methods for Determining Modifiers of Cellular Disease State

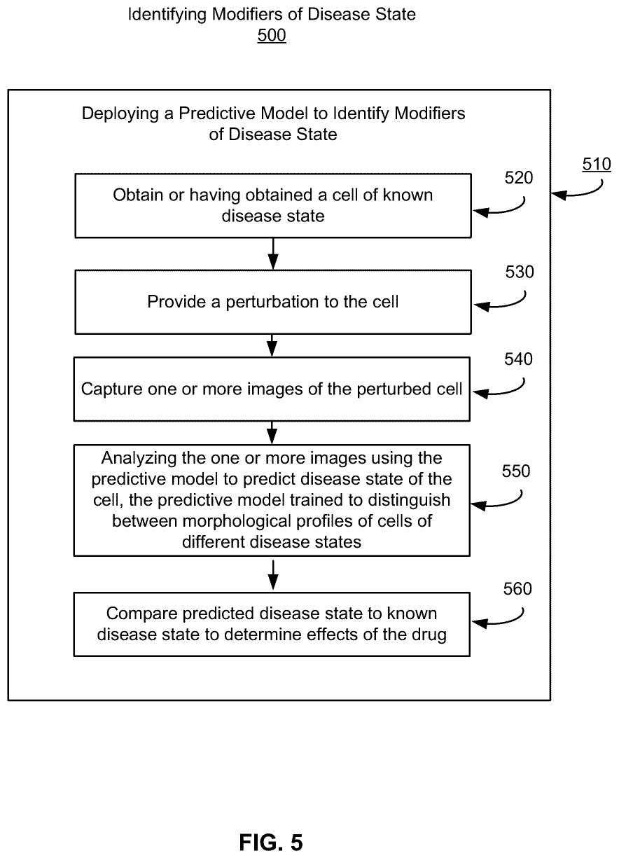

FIG. 5 is a flow process 500 for identifying modifiers of cellular disease state by deploying a predictive model, in accordance with an embodiment. For example, the predictive model may, in various embodiments, be trained using the flow process step 305 described in FIG. 3.

Here, step 510 of deploying a predictive model to identify modifiers of cellular disease state involves steps 520, 530, 540, 550, and 560. Step 520 involves obtaining or having obtained a cell of known disease state or a number of cells with a same known disease state. For example, the cell(s) may have been obtained from a subject of a known disease state. As another example, the cell(s) may have been previously analyzed by deploying a predictive model (e.g., step 355 shown in FIG. 3B) which predicted a cellular disease state for the cell(s).

Step 530 involves providing a perturbation to the cell(s). For example, the perturbation can be provided to the cell(s) within a well in a well plate (e.g., in a well of a 96 well plate). Here, the provided perturbation may have an effect on the disease state of the cell(S), which can be manifested by the cell(s) as changes in the cell morphological profile. Thus, subsequent to providing the perturbation to the cell(s), the cellular disease state of the cell(s) may no longer be known.

Step 540 involves capturing one or more images of the perturbed cell(s). As an example, the cell(s) may have been stained (e.g., with Cell Paint stains) and therefore, the different images of the cell(s) correspond to different fluorescent channels that include fluorescent intensity indicating the cell nuclei, nucleic acids, endoplasmic reticulum, actin/Golgi/plasma membrane, and mitochondria.

Step 550 involves analyzing the one or more images using the predictive model to predict the disease state of the perturbed cell(s). Here, the predictive model was previously trained to distinguish between morphological profiles of cells of different disease states. Thus, in some embodiments, the predictive model predicts a disease state of the cell(s) by comparing the morphological profile of the cell(s), including the averaged morphological profile of the number of cells, with morphological profiles of cells of known disease states.

Step 560 involves comparing the predicted cellular disease state to the previous known disease state of the cell (e.g., prior to perturbation) to determine the effects of the drug on cellular disease state. For example, if the perturbation caused the cell to exhibit morphological changes that were predicted to be less of a disease state, the perturbation can be characterized as having a therapeutic effect. As another example, if the perturbation caused the cell to exhibit morphological changes that were predicted to be a more diseased phenotype, the perturbation can be characterized as having a detrimental effect on the disease state.

### Cells

In various embodiments, the cells (e.g., cells shown in FIG. 1) refer to a single cell. In various embodiments, the cells refer to a population of cells. In various embodiments, the cells refer to multiple populations of cells. The cells can vary in regard to the type of cells (single cell type, mixture of cell types), or culture type (e.g., in vitro 2D culture, in vitro 3D culture, or ex vivo). In various embodiments, the cells include one or more cell types. In various embodiments, the cells are a single cell population with a single cell type. In various embodiments, the cells are stem cells. In various embodiments, the cells are partially differentiated cells. In various embodiments, the cells are terminally differentiated cells. In various embodiments, the cells are somatic cells. In various embodiments, the cells are fibroblasts. In various embodiments, the cells are peripheral blood mononuclear cells (PBMCs). In various embodiments, the cells include one or more of stem cells, partially differentiated cells, terminally differentiated cells, somatic cells, or fibroblasts.

In various embodiments, the cells are obtained from a subject, such as a human subject. Therefore, the disease analysis pipeline described herein can be applied to determine disease states of the cells obtained from the subject. In various embodiments, the disease analysis pipeline can be used to diagnose the subject with a disease, or to classify the subject as having a particular subtype of the disease. In various embodiments, the cells are obtained from a sample that is obtained from a subject. An example of a sample can include an aliquot of body fluid, such as a blood sample, taken from a subject, by means including venipuncture, excretion, ejaculation, massage, biopsy, needle aspirate, lavage sample, scraping, surgical incision, or intervention or other means known in the art. As another example, a sample can include a tissue sample obtained via a tissue biopsy. In particular embodiments, a tissue biopsy can be obtained from an extremity of the subject (e.g., arm or leg of the subject).

In various embodiments, the cells are seeded and cultured in vitro in a well plate. In various embodiments, the cells are seeded and cultured in any one of a 6-well plate, 12-well plate, 24-well plate, 48-well plate, 96-well plate, 384-well plate, or 1536-well plates. In particular embodiments, the cells 105 are seeded and cultured in a 96-well plate. In various embodiments, the well plates can be clear bottom well plates that enable imaging (e.g., imaging of cell stains, e.g., cell stain 150 shown in FIG. 1).

### Cell Stains

Generally, cells are treated with one or more cell stains or dyes (e.g., cell stains 150 shown in FIG. 1) for purposes of visualizing one or more aspects of cells that can be informative for determining the disease states of the cells. In particular embodiments, cell stains include fluorescent dyes, such as fluorescent antibody dyes that target biomarkers that represent known disease state hallmarks. In various embodiments, cells are treated with one fluorescent dye. In various embodiments, cells are treated with two fluorescent dyes. In various embodiments, cells are treated with three fluorescent dyes. In various embodiments, cells are treated with four fluorescent dyes. In various embodiments, cells are treated with five fluorescent dyes. In various embodiments, cells are treated with six fluorescent dyes. In various embodiments, the different fluorescent dyes used to treat cells are selected such that the fluorescent signal due to one dye minimally overlaps or does not overlap with the fluorescent signal of another dye. Thus, the fluorescent signals of multiple dyes can be imaged for a single cell.

In some embodiments, cells are treated with multiple antibody dyes, where the antibodies are specific for biomarkers that are located in different locations of the cells. For example, cells can be treated with a first antibody dye that binds to cytosolic markers and further treated with a second antibody dye that binds to nucleus markers. This enables separation of fluorescent signals arising from the multiple dyes by spatially localizing the signal from the differently located dyes.

In various embodiments, cells are treated with Cell Paint stains including stains for one or more of cell nuclei (e.g., DAPI stain), nucleoli and cytoplasmic RNA (e.g., RNA or nucleic acid stain), endoplasmic reticulum (ER stain), actin, Golgi and plasma membrane (AGP stain), and mitochondria (MITO stain). Additionally, detailed protocols of Cell Paint staining are further described in Schiff, L. et al., Deep Learning and automated Cell Painting reveal Parkinson's disease-specific signatures in primary patient fibroblasts, bioRxiv 2020.11.13.380576, which is hereby incorporated by reference in its entirety. Additional or alternative stains can include any of Alexa Fluor® 488 Conjugate (Invitrogen™ C11252), Alexa Fluor® 568 Phalloidin (Invitrogen™ A12380), Hoechst 33342 trihydrochloride, trihydrate (Invitrogen™ H3570), Molecular Probes Wheat Germ Agglutinin, or Alexa Fluor 555 Conjugate (Invitrogen™ W32464).

### Diseases and Disease States

Embodiments disclosed herein involve performing high-throughput analysis of cells using a disease analysis pipeline that determines predicted disease states of cells by implementing a predictive model trained to distinguish between morphological profiles of cells of different disease states. In various embodiments, the disease states refer to a cellular state of a particular disease.

Example diseases include, for example, a cancer, inflammatory disease, neurodegenerative disease, autoimmune disorder, neuromuscular disease, cardiac disease, or fibrotic disease.

In various embodiments, the cancer can be any one of lung bronchioloalveolar carcinoma (BAC), bladder cancer, a female genital tract malignancy (e.g., uterine serous carcinoma, endometrial carcinoma, vulvar squamous cell carcinoma, and uterine sarcoma), an ovarian surface epithelial carcinoma (e.g., clear cell carcinoma of the ovary, epithelial ovarian cancer, fallopian tube cancer, and primary peritoneal cancer), breast carcinoma, non-small cell lung cancer (NSCLC), a male genital tract malignancy (e.g., testicular cancer), retroperitoneal or peritoneal carcinoma, gastroesophageal adenocarcinoma, esophagogastric junction carcinoma, liver hepatocellular carcinoma, esophageal and esophagogastric junction carcinoma, cervical cancer, cholangiocarcinoma, pancreatic adenocarcinoma, extrahepatic bile duct adenocarcinoma, a small intestinal malignancy, gastric adenocarcinoma, cancer of unknown primary (CUP), colorectal adenocarcinoma, esophageal carcinoma, prostatic adenocarcinoma, kidney cancer, head and neck squamous carcinoma, thymic carcinoma, non-melanoma skin cancer, thyroid carcinoma (e.g., papillary carcinoma), a head and neck cancer, anal carcinoma, non-epithelial ovarian cancer (non-EOC), uveal melanoma, malignant pleural mesothelioma, small cell lung cancer (SCLC), a central nervous system cancer, a neuroendocrine tumor, and a soft tissue tumor. For example, in certain embodiments, the cancer is breast cancer, non-small cell lung cancer, bladder cancer, kidney cancer, colon cancer, and melanoma.

In various embodiments, the inflammatory disease can be any one of acute respiratory distress syndrome (ARDS), acute lung injury (ALI), alcoholic liver disease, allergic inflammation of the skin, lungs, and gastrointestinal tract, allergic rhinitis, ankylosing spondylitis, asthma (allergic and non-allergic), atopic dermatitis (also known as atopic eczema), atherosclerosis, celiac disease, chronic obstructive pulmonary disease (COPD), chronic respiratory distress syndrome (CRDS), colitis, dermatitis, diabetes, eczema, endocarditis, fatty liver disease, fibrosis (e.g., idiopathic pulmonary fibrosis, scleroderma, kidney fibrosis, and scarring), food allergies (e.g., allergies to peanuts, eggs, dairy, shellfish, tree nuts, etc.), gastritis, gout, hepatic steatosis, hepatitis, inflammation of body organs including joint inflammation including joints in the knees, limbs or hands, inflammatory bowel disease (IBD) (including Crohn's disease or ulcerative colitis), intestinal hyperplasia, irritable bowel syndrome, juvenile rheumatoid arthritis, liver disease, metabolic syndrome, multiple sclerosis, myasthenia gravis, neurogenic lung edema, nephritis (e.g., glomerular nephritis), non-alcoholic fatty liver disease (NAFLD) (including non-alcoholic steatosis and non-alcoholic steatohepatitis (NASH)), obesity, prostatitis, psoriasis, psoriatic arthritis, rheumatoid arthritis (RA), sarcoidosis sinusitis, splenitis, seasonal allergies, sepsis, systemic lupus erythematosus, uveitis, and UV-induced skin inflammation.

In various embodiments, the neurodegenerative disease can be any one of Alzheimer's disease, Parkinson's disease, traumatic CNS injury, Down Syndrome (DS), glaucoma, amyotrophic lateral sclerosis (ALS), frontotemporal dementia (FTD), and Huntington's disease. In addition, the neurodegenerative disease can also include Absence of the Septum Pellucidum, Acid Lipase Disease, Acid Maltase Deficiency, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS, Alexander Disease, Alper's Disease, Alternating Hemiplegia, Anencephaly, Aneurysm, Angelman Syndrome, Angiomatosis, Anoxia, Antiphosphipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Autism, Autonomic Dysfunction, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Injuries, Bradbury-Eggleston Syndrome, Brain or Spinal Tumors, Brain Aneurysm, Brain injury, Brown-Sequard Syndrome, Bulbospinal Muscular Atrophy, Cadasil, Canavan Disease, Causalgia, Cavernomas, Cavernous Angioma, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Chorea, Chronic Inflammatory Demyelinating Polyneuropathy (CIDP), Coffin Lowry Syndrome, Colpocephaly, Congenital Facial Diplegia, Congenital Myasthenia, Congenital Myopathy, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Dancing Eyes-Dancing Feet Syndrome, Dandy-Walker Syndrome, Dawson Disease, Dementia, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyssynergia Cerebellaris Myoclonica, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry Disease, Fahr's Syndrome, Fainting, Familial Dysautonomia, Familial Hemangioma, Familial Periodic Paralyzes, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Hemicrania Continua, Hemifacial Spasm, Hemiplegia Alterans, Hereditary Neuropathy, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydromyelia, Hypernychthemeral Syndrome, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaac's Syndrome, Joubert syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus, Lyme Disease, Machado-Joseph Disease, Macrencephaly, Melkersson-Rosenthal Syndrome, Meningitis, Menkes Disease, Meralgia Paresthetica, Metachromatic Leukodystrophy, Microcephaly, Migraine, Miller Fisher Syndrome, Mini-Strokes, Mitochondrial Myopathies, Motor Neuron Diseases, Moyamoya Disease, Mucolipidoses, Mucopolysaccharidoses, Multiple sclerosis (MS), Multiple System Atrophy, Muscular Dystrophy, Myasthenia Gravis, Myoclonus, Myopathy, Myotonia, Narcolepsy, Neuroacanthocytosis, Neurodegeneration with Brain Iron Accumulation, Neurofibromatosis, Neuroleptic Malignant Syndrome, Neurosarcoidosis, Neurotoxicity, Nevus Cavernosus, Niemann-Pick Disease, Non 24 Sleep Wake Disorder, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Perineural Cysts, Periodic Paralyzes, Peripheral Neuropathy, Periventricular Leukomalacia, Pervasive Developmental Disorders, Pinched Nerve, Piriformis Syndrome, Plexopathy, Polymyositis, Pompe Disease, Porencephaly, Postherpetic Neuralgia, Postinfectious Encephalomyelitis, Post-Polio Syndrome, Postural Hypotension, Postural Orthostatic Tachyardia Syndrome (POTS), Primary Lateral Sclerosis, Prion Diseases, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Saint Vitus Dance, Sandhoff Disease, Schizencephaly, Septo-Optic Dysplasia, Shingles, Shy-Drager Syndrome, Sjogren's Syndrome, Sleep Apnea, Sleeping Sickness, Sotos Syndrome, Spasticity, Spinal Cord Infarction, Spinal Cord Injury, Spinal Cord Tumors, Spinocerebellar Atrophy, Spinocerebellar Degeneration, Stiff-Person Syndrome, Striatonigral Degeneration, Stroke, Sturge-Weber Syndrome, SUNCT Headache, Syncope, Syphilitic Spinal Sclerosis, Syringomyelia, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tinnitus, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

In various embodiments, the autoimmune disease can be any one of: arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis deformans, polyarthritis chronica primaria, reactive arthritis, and ankylosing spondylitis; inflammatory hyperproliferative skin diseases; psoriasis, such as plaque psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic fasciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; choroiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodo sum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired splenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic reperfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis. In particular embodiments, the autoimmune disorder in the subject can include one or more of: systemic lupus erythematosus (SLE), lupus nephritis, chronic graft versus host disease (cGVHD), rheumatoid arthritis (RA), Sjogren's syndrome, vitiligo, inflammatory bowed disease, and Crohn's Disease. In particular embodiments, the autoimmune disorder is systemic lupus erythematosus (SLE). In particular embodiments, the autoimmune disorder is rheumatoid arthritis.

In particular embodiments, the disease refers to a neurodegenerative disease or any other disease that can be detected based on Cell Paint staining.

In particular embodiments, neurodegenerative diseases include any of Parkinson's Disease (PD), Alzheimer's Disease, Amyotrophic Lateral Sclerosis (ALS), Infantile Neuroaxonal Dystrophy (INAD), Multiple Sclerosis (MS), Amyotrophic Lateral Sclerosis (ALS), Batten Disease, Charcot-Marie-Tooth Disease (CMT), Autism, post traumatic stress disorder (PTSD), schizophrenia, frontotemporal dementia (FTD), multiple system atrophy (MSA), or a synucleinopathy.

In various embodiments, the disease state refers to one of a presence or absence of a disease. For example, in the context of Parkinson's disease (PD), the disease state refers to a presence or absence of PD. In various embodiments, the disease state refers to a subtype of a disease. For example, in the context of Parkinson's disease, the disease state refers to one of an LRRK2 subtype, a GBA subtype, or a sporadic subtype. For example, in the context of Charcot-Marie-Tooth Disease (CMT), the disease state refers to one of a CMT1A subtype, CMT2B subtype, CMT4C subtype, or CMTX1 subtype.

### Perturbations

One or more perturbations (e.g., perturbation 160 shown in FIG. 1) can be provided to cells. In various embodiments, a perturbation can be a small molecule drug from a library of small molecule drugs. In various embodiments, a perturbation is a drug or compound that is known to have disease-state modifying effects, examples of which include Levodopa based drugs, Carbidopa based drugs, dopamine agonists, catechol-O-methyltransferase (COMT) inhibitors, monoamine oxidase (MAO) inhibitors, Rho-kinase inhibitors, A2A receptor antagonists, dyskinesia treatments, anticholinergics, and acetylocholinesterase inhibitors, which have been shown to have anti-aging effects. Examples of dopamine agonists include pramipexole (MIRAPEX), Ropinirole (REQUIP), Rotigotine (NEUPRO), apomorphine HCl (KYNMOBI). Examples of COMT inhibitors include Opicapone (ONGENTYS), Entacapone (COMTAN), and Tolcapone (TASMAR). Examples of MAO inhibitors include selegiline (ELDEPRYL or ZELAPAR), Rasagiline (AZILECT or AZIPRON), and safinamide (XADAGO). An example of a Rho-kinase inhibitor includes Fasudil. An example of A2A receptor antagonists includes Istradefylline (NOURIANZ). Examples of dyskinesia treatments include Amantadine ER (GOCOVRI, SYMADINE, or SYMMETREL) and Pridopidine (HUNTEXIL). Examples of anticholinergics include benztropine mesylate (COGENTIN) and trihexyphenidyl (ARTANE). An example of acetylcholinesterase inhibitors includes rivastigmine (EXELON).

In various embodiments, the perturbation is any one of bafilomycin, carbonyl cyanide m-chlorophenyl hydrazone (CCCP), MGA312, rotenone, or valinomycin. In particular embodiments, the perturbation is bafilomycin. In particular embodiments, the perturbation is CCCP. In particular embodiments, the perturbation is MGA312. In particular embodiments, the perturbation is rotenone. In particular embodiments, the perturbation is valinomycin.

In various embodiments, a perturbation is provided to cells that are seeded and cultured within a well in a well plate. In particular embodiments, a perturbation is provided to cells within a well through an automated, high-throughput process. In various embodiments, a perturbation is applied to cells at a concentration between 0.1-100,000 nM. In various embodiments, a perturbation is applied to cells at a concentration between 1-10,000 nM. In various embodiments, a perturbation is applied to cells at a concentration between 1-5,000 nM. In various embodiments, a perturbation is applied to cells at a concentration between 1-2,000 nM. In various embodiments, a perturbation is applied to cells at a concentration between 1-1,000 nM. In various embodiments, a perturbation is applied to cells at a concentration between 1-500 nM. In various embodiments, a perturbation is applied to cells at a concentration between 1-250 nM. In various embodiments, a perturbation is applied to cells at a concentration between 1-100 nM. In various embodiments, a perturbation is applied to cells at a concentration between 1-50 nM. In various embodiments, a perturbation is applied to cells at a concentration between 1-20 nM. In various embodiments, a perturbation is applied to cells at a concentration between 1-10 nM. In various embodiments, a perturbation is applied to cells at a concentration between 10-50,000 nM. In various embodiments, a perturbation is applied to cells at a concentration between 10-10,000 Mn. In various embodiments, a perturbation is applied to cells at a concentration between 10-1,000 nM. In various embodiments, a perturbation is applied to cells at a concentration between 10-500M. In various embodiments, a perturbation is applied to cells at a concentration between 100-1000 nM. In various embodiments, a perturbation is applied to cells at a concentration between 200-1000 nM. In various embodiments, a perturbation is applied to cells at a concentration between 500-1000 nM. In various embodiments, a perturbation is applied to cells at a concentration between 300-2000 nM. In various embodiments, a perturbation is applied to cells at a concentration between 350-1600 nM. In various embodiments, a perturbation is applied to cells at a concentration between 500-1200 nM.

In various embodiments, a perturbation is applied to cells at a concentration between 1-100 μM. In various embodiments, a perturbation is applied to cells at a concentration between 1-50 μM. In various embodiments, a perturbation is applied to cells at a concentration between 1-25 μM. In various embodiments, a perturbation is applied to cells at a concentration between 5-25 μM. In various embodiments, a perturbation is applied to cells at a concentration between 10-15 μM. In various embodiments, a perturbation is applied to cells at a concentration of about 1 μM. In various embodiments, a perturbation is applied to cells at a concentration of about 5 μM. In various embodiments, a perturbation is applied to cells at a concentration of about 10 μM. In various embodiments, a perturbation is applied to cells at a concentration of about 15 μM. In various embodiments, a perturbation is applied to cells at a concentration of about 20 μM. In various embodiments, a perturbation is applied to cells at a concentration of about 25 μM. In various embodiments, a perturbation is applied to cells at a concentration of about 40 μM. In various embodiments, a perturbation is applied to cells at a concentration of about 50 μM.

In various embodiments, a perturbation is applied to cells for at least 30 minutes. In various embodiments, a perturbation is applied to cells for at least 1 hour. In various embodiments, a perturbation is applied to cells for at least 2 hours. In various embodiments, a perturbation is applied to cells for at least 3 hours. In various embodiments, a perturbation is applied to cells for at least 4 hours. In various embodiments, a perturbation is applied to cells for at least 6 hours. In various embodiments, a perturbation is applied to cells for at least 8 hours. In various embodiments, a perturbation is applied to cells for at least 12 hours. In various embodiments, a perturbation is applied to cells for at least 18 hours. In various embodiments, a perturbation is applied to cells for at least 24 hours. In various embodiments, a perturbation is applied to cells for at least 36 hours. In various embodiments, a perturbation is applied to cells for at least 48 hours. In various embodiments, a perturbation is applied to cells for at least 60 hours. In various embodiments, a perturbation is applied to cells for at least 72 hours. In various embodiments, a perturbation is applied to cells for at least 96 hours. In various embodiments, a perturbation is applied to cells for at least 120 hours. In various embodiments, a perturbation is applied to cells for between 30 minutes and 120 hours. In various embodiments, a perturbation is applied to cells for between 30 minutes and 60 hours. In various embodiments, a perturbation is applied to cells for between 30 minutes and 24 hours. In various embodiments, a perturbation is applied to cells for between 30 minutes and 12 hours. In various embodiments, a perturbation is applied to cells for between 30 minutes and 6 hours. In various embodiments, a perturbation is applied to cells for between 30 minutes and 4 hours. In various embodiments, a perturbation is applied to cells for between 30 minutes and 2 hours.

### Imaging Device

The imaging device (e.g., imaging device 120 shown in FIG. 1) captures one or more images of the cells which are analyzed by the predictive model system 130. The cells may be cultured in an, e.g., in vitro 2D culture, in vitro 3D culture, or ex vivo. Generally, the imaging device is capable of capturing signal intensity from dyes (e.g., cell stains 150) that have been applied to the cells. Therefore, the imaging device captures one or more images of the cells including signal intensity originating from the dyes. In particular embodiments, the dyes are fluorescent dyes and therefore, the imaging device captures fluorescent signal intensity from the dyes. In various embodiments, the imaging device is any one of a fluorescence microscope, confocal microscope, or two-photon microscope.

In various embodiments, the imaging device captures images across multiple fluorescent channels, thereby delineating the fluorescent signal intensity that is present in each image. In one scenario, the imaging device captures images across at least 2 fluorescent channels. In one scenario, the imaging device captures images across at least 3 fluorescent channels. In one scenario, the imaging device captures images across at least 4 fluorescent channels. In one scenario, the imaging device captures images across at least 5 fluorescent channels.

In various embodiments, the imaging device captures one or more images per well in a well plate that includes the cells. In various embodiments, the imaging device captures at least 1 tile per well in the well plates. In various embodiments, the imaging device captures at least 10 tiles per well in the well plates. In various embodiments, the imaging device captures at least 15 tiles per well in the well plates. In various embodiments, the imaging device captures at least 20 tiles per well in the well plates. In various embodiments, the imaging device captures at least 25 tiles per well in the well plates. In various embodiments, the imaging device captures at least 30 tiles per well in the well plates. In various embodiments, the imaging device captures at least 35 tiles per well in the well plates. In various embodiments, the imaging device captures at least 40 tiles per well in the well plates. In various embodiments, the imaging device captures at least 45 tiles per well in the well plates. In various embodiments, the imaging device captures at least 50 tiles per well in the well plates. In various embodiments, the imaging device captures at least 75 tiles per well in the well plates. In various embodiments, the imaging device captures at least 100 tiles per well in the well plates. Therefore, in various embodiments, the imaging device captures numerous images per well plate. For example, the imaging device can capture at least 100 images, at least 1,000 images, or at least 10,000 images from a well plate. In various embodiments, when the high-throughput disease prediction system 140 is implemented over numerous well plates and cell lines, at least 100 images, at least 1,000 images, at least 10,000 images, at least 100,000 images, or at least 1,000,000 images are captured for subsequent analysis.

In various embodiments, imaging device may capture images of cells over various time periods. For example, the imaging device may capture a first image of cells at a first timepoint and subsequently capture a second image of cells at a second timepoint. In various embodiments, the imaging device may capture a time lapse of cells over multiple time points (e.g., over hours, over days, or over weeks). Capturing images of cells at different time points enables the tracking of cell behavior, such as cell mobility, which can be informative for predicting the ages of different cells. In various embodiments, to capture images of cells across different time points, the imaging device may include a platform for housing the cells during imaging, such that the viability of the cultured cells is not impacted during imaging. In various embodiments, the imaging device may have a platform that enables control over the environment conditions (e.g., O₂ or CO₂ content, humidity, temperature, and pH) that are exposed to the cells, thereby enabling live cell imaging.

System and/or Computer Embodiments

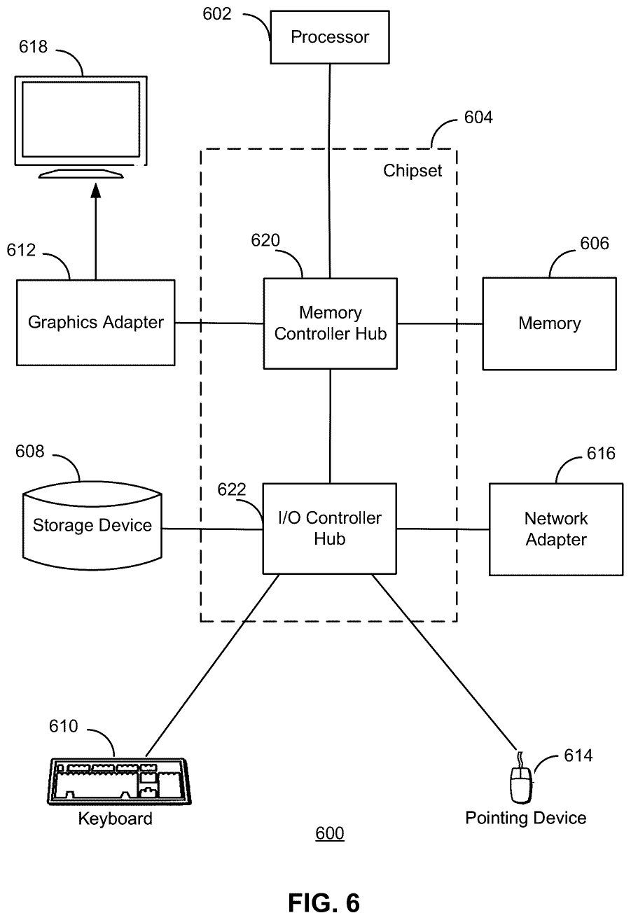

FIG. 6 depicts an example computing device 600 for implementing system and methods described in reference to FIGS. 1-5. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like. In various embodiments, the computing device 600 can operate as the predictive model system 130 shown in FIG. 1 (or a portion of the predictive model system 130). Thus, the computing device 600 may train and/or deploy predictive models for predicting disease states of cells.

In some embodiments, the computing device 600 includes at least one processor 602 coupled to a chipset 604. The chipset 604 includes a memory controller hub 620 and an input/output (I/O) controller hub 622. A memory 606 and a graphics adapter 612 are coupled to the memory controller hub 620, and a display 618 is coupled to the graphics adapter 612. A storage device 608, an input interface 614, and network adapter 616 are coupled to the I/O controller hub 622. Other embodiments of the computing device 600 have different architectures.

The storage device 608 is a non-transitory computer-readable storage medium such as a hard drive, compact disk read-only memory (CD-ROM), DVD, or a solid-state memory device. The memory 606 holds instructions and data used by the processor 602. The input interface 614 is a touch-screen interface, a mouse, track ball, or other types of input interface, a keyboard, or some combination thereof, and is used to input data into the computing device 600. In some embodiments, the computing device 600 may be configured to receive input (e.g., commands) from the input interface 614 via gestures from the user. The graphics adapter 612 displays images and other information on the display 618. The network adapter 616 couples the computing device 600 to one or more computer networks.

The computing device 600 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 608, loaded into the memory 606, and executed by the processor 602.

The types of computing devices 600 can vary from the embodiments described herein. For example, the computing device 600 can lack some of the components described above, such as graphics adapters 612, input interface 614, and displays 618. In some embodiments, a computing device 600 can include a processor 602 for executing instructions stored on a memory 606.

The methods disclosed herein can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

### ADDITIONAL EMBODIMENTS

The present disclosure describes combining advances in machine learning and scalable automation, to develop an automated high-throughput screening system for the morphology-based profiling of a neurodegenerative disease or other diseases, which allows to determine a disease-specific cell phenotype or cell signature, and to allow to predict the disease state of cells with an unknown disease state. The system includes a cell culture unit for culturing cells, and an imaging system operable to generate images of the cells and analyze the images of the cells. The imaging system includes a computer processor having instructions for identifying a disease-specific cell phenotype, such as disease-specific morphological features of the cells based on the cell images. The system includes a predictive model pre-trained for identifying a disease-specific cell phenotype by comparing morphological features of cells of a disease state with morphological features of cells of a non-disease state.

The imaging system also includes instructions for predicting the disease state of a subject. In various embodiments, the predictive model is trained using cells with known disease states. In particular embodiments, the predictive model is trained using combined morphological profiles of synthetic pools of known disease states. To predict the disease state of a subject, the morphological profile of cells from the subject of unknown disease state is input into the trained predictive model, which then compared the morphological profile of the cells of unknown state with the morphological profiles of known disease states, to determine the disease state of the subject.

Embodiments disclosed herein also provide an automated method for analyzing cells which includes culturing cells and analyzing the cultured cells using the system of the present disclosure. In various embodiments, the analyzing of the cultured cells includes the determination of disease-specific cell phenotype or cell signature and prediction of the unknown disease state of a subject using the predictive model.

Additionally disclosed herein is an automated method for screening putative therapeutic agents. The method includes culturing cells having a disease-specific signature, contacting the cell with a putative therapeutic agent or an exogenous stressor, and analyzing the cells and identifying a change in the disease-specific signature caused by the putative therapeutic agent or the exogenous stressor, thereby performing automated screening of potential therapeutic agents for the disease.

In various embodiments, a predictive model is applied to the disclosed systems and methods for identifying the disease-specific cell phenotype or cell signature, predicting the disease state of a subject, and screening putative therapeutic agents. In various embodiments, the predictive model is trained based on the morphological profiles of cells of known disease states. In particular embodiments, the predictive model is trained based on morphological profiles of cells from a synthetic pool that includes cells randomly selected from cell lines of different donors. For example, the predictive model can be trained based on combined morphological profile of the randomly selected cells from the synthetic pool.

In various embodiments, the predictive model trained by using the synthetic pool has advantages when compared to a predictive model trained by using morphological profiles from single cells without pooling the cells and averaging the morphological profiles. By averaging the cells' information from a plurality of sources (e.g., different donors), the source-specific variations (e.g., donor-specific features) can be smoothened, which then allows the state-specific features (e.g., disease-specific features) to be highlighted when training the predictive model. In the following, the exemplary applications of the predictive model that artificially pool together single cells from different donors with a common disease state are further illustrated.

### EXAMPLES

### Example 1: Example Disease Analysis Pipeline

Disclosed herein is an automated platform to morphologically profile collections of cells leveraging the cell culture automation capabilities of the New York Stem Cell Foundation (NYSCF) Global Stem Cell Array®, a modular robotic platform for large-scale cell culture automation. The NYSCF Global Stem Cell Array was applied to search for disease-specific cell features, which is also referred to as disease-specific cell signature or cell phenotype or simply disease signature or disease phenotype.

Taking the INAD disease as an example, starting from a collection of cell lines in the NYSCF repository that were collected from different subjects and derived using highly standardized methods, an automated experimental procedure was applied in a high-content profiling platform to generate predictions of a presence or absence of an INAD disease state in cells. The automated experimental procedure includes an image analysis pipeline that operates on the INAD cell lines and healthy controls to generate morphological profiles that distinguish between healthy and INAD cells. In particular embodiments, a deep metric network (DMN) that maps each whole or cell crop image independently to an embedding vector, which, along with CellProfiler features and basic image statistics, are used as data sources for model fitting and evaluation for various supervised prediction tasks. The automated procedures were designed to minimize experimental variation and maximize reproducibility across plates, which resulted in consistent growth of prediction probabilities at both cell-line level and well level.

Methods

Staining and imaging. To fluorescently label the cells, the protocol published in Bray et al. was adapted to an automated liquid handling system (Hamilton STAR). Briefly, plates were placed on deck for addition of culture medium containing MitoTracker (Invitrogen™ M22426) and incubated at 37° C. for 30 minutes, then cells were fixed with 4% Paraformaldehyde (Electron Microscopy Sciences, 15710-S), followed by permeabilization with 0.1% Triton X-100 (Sigma-Aldrich, T8787) in 1×HBSS (Thermo Fisher Scientific, 14025126). After a series of washes, cells were stained at room temperature with the Cell Painting staining cocktail for 30 minutes, which contains Concanavalin A, Alexa Fluor® 488 Conjugate (Invitrogen™ C11252), SYTO® 14 Green Fluorescent Nucleic Acid Stain (Invitrogen™ S7576), Alexa Fluor® 568 Phalloidin (Invitrogen™ A12380), Hoechst 33342 trihydrochloride, trihydrate (Invitrogen™ H3570), Molecular Probes Wheat Germ Agglutinin, Alexa Fluor 555 Conjugate (Invitrogen™ W32464). Plates were washed twice and imaged immediately.

The images were acquired using an automated epifluorescence system (Nikon Ti2). For each of the wells acquired per plate, the system performed an autofocus task in the ER channel, which provided dense texture for contrast, in the center of the well, and then acquired non-overlapping tiles per well at a 40× magnification (Olympus CFI-60 Plan Apochromat Lambda 0.95 NA). To capture the entire Cell Painting panel, 5 different combinations of excitation illumination (SPECTRA X from Lumencor) and emission filters (395 nm and 447/60 nm for Hoechst, 470 nm and 520/28 nm for Concanavalin A, 508 nm and 593/40 nm for RNA-SYTO14, 555 nm and 640/40 nm for Phalloidin and wheat-germ agglutinin, and 640 nm and 692/40 nm for MitoTracker Deep Red) were used. Each 16-bit 5056×2960 tile image was acquired using NIS-Elements AR acquisition software from the image sensor (Photometrics Iris 15, 4.25 μm pixel size). Each well plate resulted in approximately 1 terabyte of data.

Image statistics features. For assessing data quality and baseline predictive performance on classification tasks, various image statistics were computed. Statistics are computed independently for each of the 5 channels for the image crops centered on detected cell objects. For each tile or cell, a “focus score” between 0.0 and 1.0 was assigned using a pre-trained deep neural network model. Otsu's method was used to segment the foreground pixels from the background and the mean and standard deviation of both the foreground and background were calculated. Foreground fraction was calculated as the number of foreground pixels divided by the total pixels. All features were normalized by subtracting the mean of each batch and plate layout from each feature and then scaling each feature to have unit L2 norm across all examples.

Image pre-processing. 16-bit images were flat field-corrected. Next, Otsu's method was used in the DAPI channel to detect nuclei centers. Images were converted to 8-bit after clipping at the 0.001 and 1.0 minimum and maximum percentile values per channel and applying a log transformation. These 8-bit 5056×2960×5 images, along with 512×512×5 image crops centered on the detected nuclei, were used to compute deep embeddings. Only image crops existing entirely within the original image boundary were included for deep embedding generation.

Deep image embedding generation. Deep image embeddings were computed on both the tile images and the 512×512×5 cell image crops. In each case, for each image and each channel independently, the single channel image was duplicated across the RGB (red-green-blue) channels and then inputted the 512×512×3 image into an Inception architecture convolutional neural network, pre-trained on the ImageNet object recognition dataset consisting of 1.2 million images of a thousand categories of (non-cell) objects, and then extracted the activations from the penultimate fully connected layer and took a random projection to get a 64-dimensional deep embedding vector (i.e., 64×1×1). The five vectors from the 5 image channels were concatenated to yield a 320-dimensional vector or embedding for each tile or cell crop. 0.7% of tiles were omitted because they were either in wells never plated with cells due to shortages or because no cells were detected, yielding a final dataset consisting of 347,821 tile deep embeddings and 5,813,995 cell image deep embeddings. All deep embeddings were normalized by subtracting the mean of each batch and plate layout from each deep embedding. Finally, datasets of the well-mean deep embeddings were computed, the mean across all cell or tile deep embeddings in a well, for all wells.

CellProfiler feature generation. A CellProfiler pipeline template was used which determined Cells in the RNA channel, Nuclei in the DAPI channel and Cytoplasm by subtracting the Nuclei objects from the Cell objects. CellProfiler version 3.1.5 was ran independently on each 16-bit 5056×2960×5 tile image set, inside a Docker container on Google Cloud. 0.2% of the tiles resulted in errors after multiple attempts and were omitted. Features were concatenated across Cells, Cytoplasm and Nuclei to obtain a 3483-dimensional feature vector per cell, across 7,450,738 cells. A reduced dataset was computed with the well-mean feature vector per well. All features were normalized by subtracting the mean of each batch and plate layout from each feature and then scaling each feature to have unit L2 norm across all examples.

Modeling and analysis. Several classification tasks were evaluated ranging from cell line prediction to disease state prediction using various data sources and multiple classification models. Data sources consisted of image statistics, CellProfiler features and deep image embeddings. Since data sources and predictions could have existed at different levels of aggregation ranging from the cell-level, tile-level, well-level to cell line-level, well-mean aggregated data sources (i.e., averaging all cell features or tile embeddings in a well) were used as input to all classification models, and aggregated the model predictions by averaging predicted probability distributions (i.e., the cell line-level prediction, by averaging predictions across wells for a cell line). In each classification task, an appropriate cross-validation approach was defined and all figures of merit reported are those on the held-out test sets. For example, the well-level accuracy is the accuracy of the set of model predictions on the held-out wells, and the cell line-level accuracy is the accuracy of the set of cell line-level predictions from held-out wells. The former indicates the expected performance with just one well example, while the latter indicates expected performance from averaging predictions across multiple wells; any gap could be due to intrinsic biological, process or modeling noise and variation.

Various classification models (sklearn) were used, including a cross-validated logistic regression (solver=“lbfgs”, max_iter=1000000), random forest classifier (with 100 base estimators), cross-validated ridge regression and multilayer perceptron (single hidden layer with 200 neurons, max_iter=1000000); these settings ensured solver convergence to the default tolerance.

Cell line identification analysis. For each of the various data sources, the cross-validation sets were utilized. For each train/test split, one of several classification models was fit or trained to predict a probability distribution across the unique cell lines and wells. For each prediction, both the top predicted cell line, the cell line class to which the model assigns the highest probability, as well as the predicted rank, the rank of probability assigned to the true cell line (i.e., when the top predicted cell line is the correct one, the predicted rank is 1) were evaluated. As the figure of merit, the well-level or cell line-level accuracy, the fraction of wells or cell Lines for which the top predicted cell line among the 96 possible choices was correct, was used.

### Example 2: Successfully Differentiating Healthy State and INAD Disease State

The predictive model for differentiating healthy state and INAD disease state was first trained using a synthetic pool that included cells randomly selected from different cell lines from different donors. The synthetic pool was created by pooling together different cell lines from different donors. The synthetic pool was not necessarily a physical pooling of randomly selected cells together, but rather a “pooling” of images or transformed representations of images obtained from cells randomly selected from different cell lines. By “pooling” the images or transformed representations of images, it means that the transformed representations of images are considered as a whole, for example, to obtain an averaged morphological profile representing the pool (or representing the morphological profiles of the cells randomly selected from different cell lines from different donors).

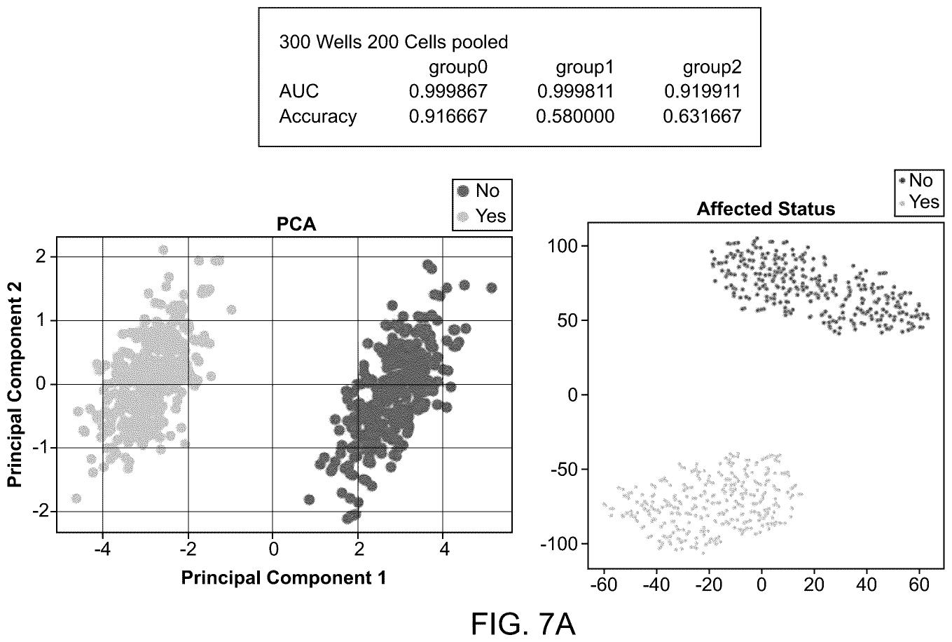

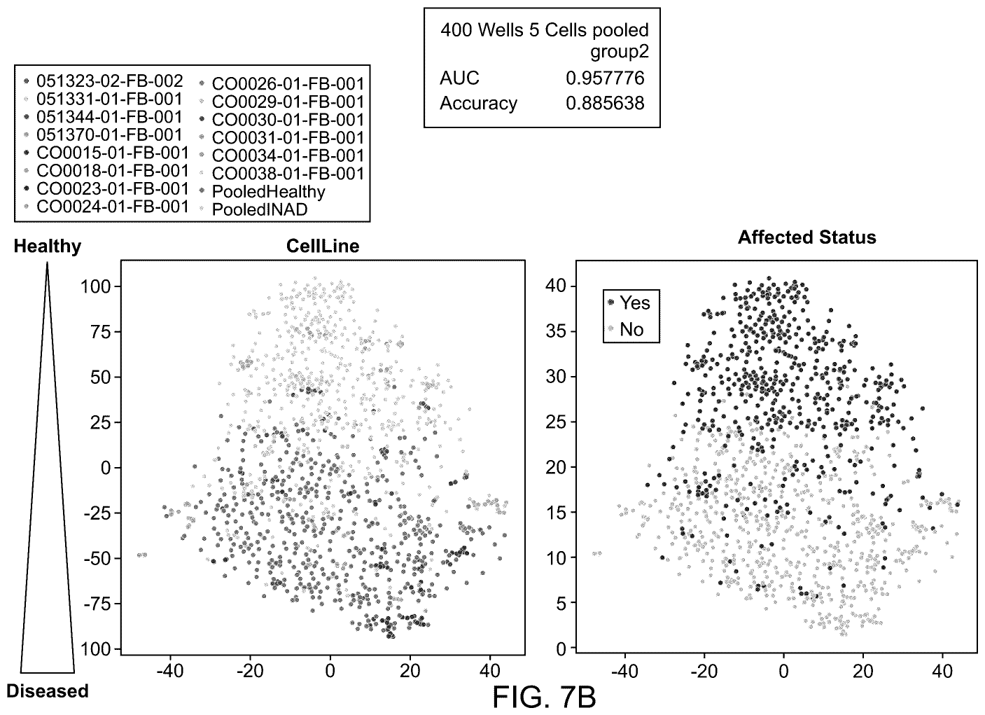

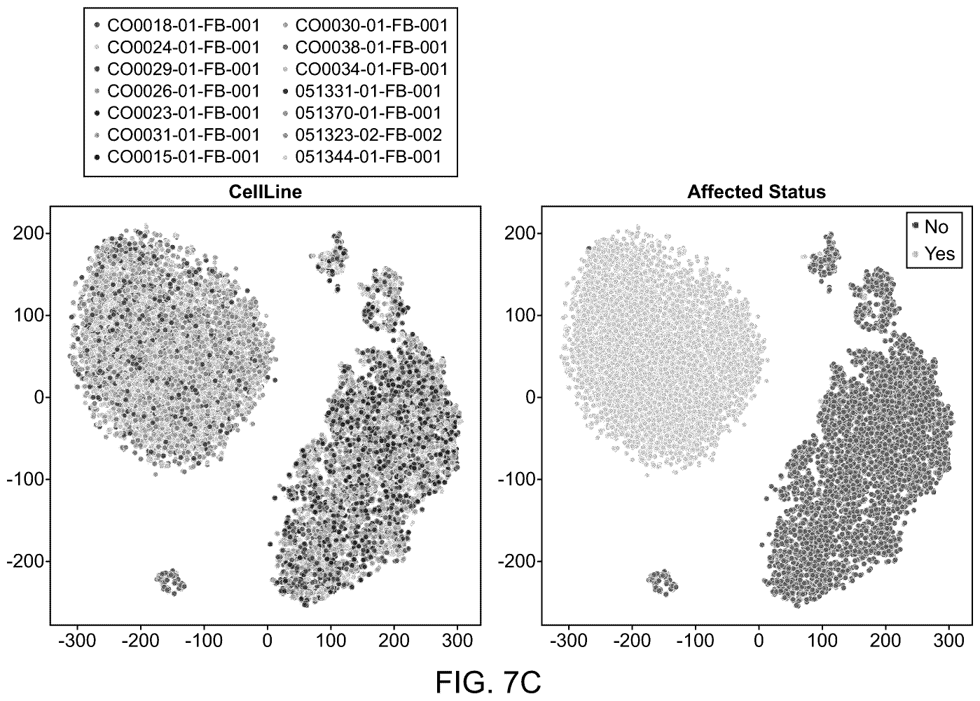

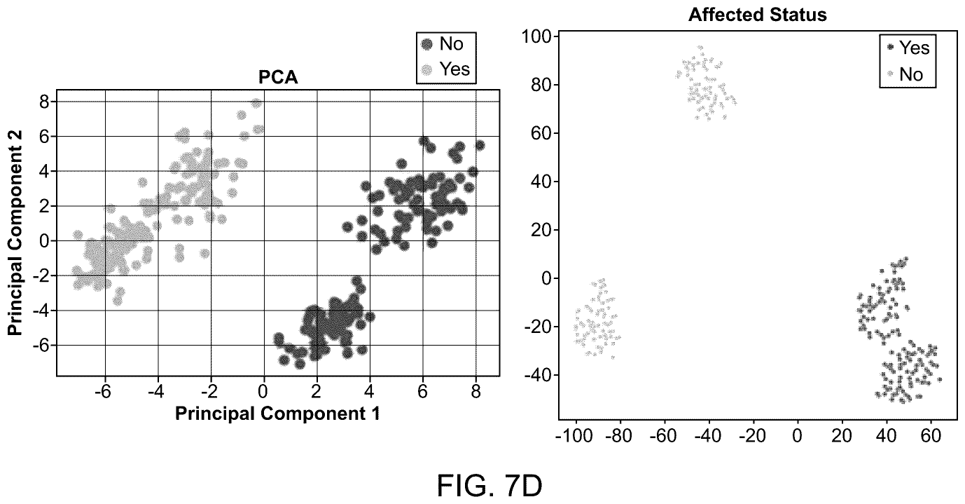

Here, the synthetic pool included images or transformed representations of images of randomly selected cells obtained from different time points. These randomly selected cells from different cell lines from different donors shared a common known disease state, which then allowed them to be pooled as a representation of the disease state during the supervised training of the predictive model. The as-trained predictive model performs well in differentiating unknown cells between healthy state and INAD disease state, as further illustrated below with reference to FIGS. 7A-7D.

FIG. 7A illustrates an example performance of the predictive model trained by pooling the morphological profiles of both training and testing datasets. In one example, a dataset containing 9 cell lines from healthy donors and 9 cell lines from diseased donors were collected. The dataset was divided into 3 groups or 3 folds (3 healthy and 3 disease cell lines per fold), which were then used for cross-validation in training and testing the predictive model. Among the three folds, two folds (6 pairs of healthy and diseased cell lines) were pooled together for creating the synthetic pools for training purposes. The predictive model was trained on the synthetic wells created from the pooled two folds on a binary classification task, healthy vs. INAD, before testing the model on the held-out fold of cell line pairs (3 pairs of healthy and diseased cell lines used to create the held-out pool). The model predictions on the held-out group were used to compute a receiver operator characteristic (ROC) curve, for which the area under the curve (ROC AUC) was evaluated. The ROC curve is the true positive rate vs. false positive rate, evaluated at different predicted probability thresholds. ROC AUC can be interpreted as the probability of correctly ranking a random healthy control and INAD cell line. The ROC AUC was computed for cell line-level predictions, the average of the models' predictions for each well from each cell line. Part (a) of FIG. 7A illustrates an outcome in terms of ROC for the performance of the predictive model trained on the synthetic wells and tested on the held-out fold. As can be seen, the AUC values for each of the three groups are 0.999867, 0.999811, and 0.919911. This means the predictive model trained on the synthetic pools performed well in differentiating healthy state and INAD disease state.

Part (b) of FIG. 7A illustrates an outcome distinguishing between cell populations in terms of principal component analysis (PCA) and Part (c) in terms of TSNe for the predictive model trained and tested using synthetic pools. In the training and testing process, a dataset used to train and test the predictive model was divided by the test/train split. The procedure included taking the dataset and dividing it into two subsets. The first subset was used to fit the predictive model as the training dataset. The second subset was not used to train the model. Instead, the input element of the dataset was provided to the model. Predictions were then made and compared to the expected values. Here, the predictive model included dimensionality reduction components such as the PCA component and the TSNe for visualizing data or the outcome of differentiating healthy state and INAD disease state. As can be seen from Part (b) of FIG. 7A from the PCA component analysis and Part (c) of FIG. 7A for the TSNe analysis, the healthy and diseased populations were well clustered by the predictive model that was trained and tested on the synthetic wells or synthetic pools.

FIG. 7B illustrates another example performance of the predictive model trained by pooling the morphological profiles for training but testing on mean well values. That is, the training dataset was created by synthetically pooling different cell lines from different donors, while testing was performed on a single cell-line level by using the mean well values of each single cell line. Part (a) of FIG. 7B illustrates an outcome in terms of ROC for the performance of the predictive model trained and tested as described. It can be seen that the AUC value for the predictive model is 0.957776 (still over 0.95), which means that the predictive model still performed well when the model was trained on the synthetic pool but the testing samples were not pooled from different cell lines.

Table 1 below illustrates further performance broken down for the predictive model at the cell-line level. As can be seen from the table, in total, there were 14 cell lines tested by the trained predictive model. Among the 14 cell lines, the predictive model performed well in differentiating healthy state and INAD disease state on 13 cell lines, with the only exception being CELL LINE 009.

TABLE 1
Broken down of the cell-level performance of the
predictions for the predictive model trained using
synthetic pools but testing on mean well values.

| Cell line # | Pred | True | Predictions |
|---|---|---|---|
| CELL LINE 001 | 0.007674 | 0.0 | 0.000000 |
| CELL LINE 002 | 0.010283 | 0.0 | 0.011905 |
| CELL LINE 003 | 0.019764 | 0.0 | 0.000000 |
| CELL LINE 004 | 0.031505 | 0.0 | 0.000000 |
| CELL LINE 005 | 0.246824 | 0.0 | 0.229167 |
| CELL LINE 006 | 0.270928 | 0.0 | 0.233333 |
| CELL LINE 007 | 0.291145 | 0.0 | 0.263889 |
| CELL LINE 008 | 0.354554 | 0.0 | 0.333333 |
| CELL LINE 009 | 0.460300 | 1.0 | 0.450000 |
| CELL LINE 010 | 0.934381 | 1.0 | 0.928571 |
| CELL LINE 011 | 0.966430 | 1.0 | 1.000000 |
| CELL LINE 012 | 0.967843 | 1.0 | 0.983333 |
| CELL LINE 013 | 0.979850 | 1.0 | 0.986111 |
| CELL LINE 014 | 0.983114 | 1.0 | 1.000000 |

In the table, the values of 0.0 and 1.0 for the “True” column represent the ground truths, where 0.0 indicates a cell line is in a healthy state, while 1.0 indicates a cell line is in a disease state. The values in the “Pred” and “Predictions” column indicate the predicted probability of a cell line in a disease state. A value of over 0.5 indicates that the corresponding cell line is predicted to be more likely in a disease state, while a value of less than 0.5 indicates that the corresponding cell line is predicted to be more likely in a healthy state. The “Preds” and “Predictions” are two kinds of predictions generated by the predictive model.

Part (b) and Part (c) of FIG. 7B further illustrates the performance of the predictive model. As can be seen from Part (b), among the 14 cell lines, 8 cell lines, including CELL LINE 001, CELL LINE 002, CELL LINE 003, CELL LINE 004, CELL LINE 005, CELL LINE 006, CELL LINE 007, CELL LINE 008, aligned well with other pooled healthy cell lines, while 5 cell lines, including CELL LINE 010, CELL LINE 011, CELL LINE 012, CELL LINE 013, CELL LINE 014, aligned well with other pooled diseased cell lines. Part (c) of FIG. 7B further illustrates the separation of the healthy cells and diseased cells. From the two parts, it can be seen that the predictive model trained on the synthetic wells also performed well on the cell-line level without requiring the pooling of different cell lines in the testing dataset.

Tables 2 and 3 illustrate another example performance of the predictive model trained by pooling the morphological profiles for training but testing on mean well values. It is well known that lung cells are difficult to differentiate between healthy state and INAD disease state using machine learning-based predictive models. When lung cells were included in the synthetic pool for training, the performance of the trained prediction model did not perform well, as can be seen in Table 2. However, after lung cells were removed, the trained predictive model performed quite well in predicting different cell lines, as can be seen from the prediction values in Table 3. The overall performance of the predictive model in terms of AUC was increased from 0.960092 to 0.973448 when lung cells were removed.

TABLE 2
Broken down of the cell-level performance of the predictions for the
predictive model trained using synthetic pools including lung cells.

| Cell line # | Pred | True | Predictions |
|---|---|---|---|
| CELL LINE 002 | 0.223709 | 0.0 | 0.222824 |
| CELL LINE 003 | 0.286524 | 0.0 | 0.286474 |
| CELL LINE 001 | 0.312609 | 0.0 | 0.313036 |
| CELL LINE 004 | 0.329322 | 0.0 | 0.328013 |
| CELL LINE 005 | 0.394358 | 0.0 | 0.393902 |
| CELL LINE 015 | 0.396694 | 1.0 | 0.394520 |
| CELL LINE 016 | 0.410606 | 1.0 | 0.412862 |
| CELL LINE 007 | 0.419150 | 0.0 | 0.418241 |
| CELL LINE 006 | 0.420967 | 0.0 | 0.420859 |
| CELL LINE 008 | 0.435782 | 0.0 | 0.435439 |
| CELL LINE 009 | 0.436180 | 1.0 | 0.438062 |
| CELL LINE 012 | 0.535373 | 1.0 | 0.535787 |
| CELL LINE 011 | 0.557088 | 1.0 | 0.556882 |
| CELL LINE 010 | 0.583674 | 1.0 | 0.582573 |
| CELL LINE 013 | 0.597094 | 1.0 | 0.597859 |
| CELL LINE 014 | 0.675557 | 1.0 | 0.676261 |

TABLE 3
Broken down of the cell-level performance of the predictions for the
predictive model trained using synthetic pools excluding lung cells.

| Cell line # | Pred | True | Predictions |
|---|---|---|---|
| CELL LINE 001 | 0.000030 | 0.0 | 0.000000 |
| CELL LINE 002 | 0.000000 | 0.0 | 0.000000 |
| CELL LINE 003 | 0.002166 | 0.0 | 0.000000 |
| CELL LINE 004 | 0.016051 | 0.0 | 0.000000 |
| CELL LINE 007 | 0.254439 | 0.0 | 0.291667 |
| CELL LINE 006 | 0.320327 | 0.0 | 0.300000 |
| CELL LINE 008 | 0.322396 | 0.0 | 0.285714 |
| CELL LINE 005 | 0.342650 | 0.0 | 0.357143 |
| CELL LINE 009 | 0.408094 | 1.0 | 0.437500 |
| CELL LINE 011 | 0.979580 | 1.0 | 1.000000 |
| CELL LINE 012 | 0.988693 | 1.0 | 1.000000 |
| CELL LINE 010 | 0.991317 | 1.0 | 1.000000 |
| CELL LINE 013 | 0.997571 | 1.0 | 1.000000 |
| CELL LINE 014 | 0.999361 | 1.0 | 1.000000 |

FIG. 7C illustrates another example performance of the predictive model trained by pooling the morphological profiles for training but testing on single cell value. That is, a dataset containing all the cell lines pooled together was used as the training dataset, and the testing was performed at the single-cell level by testing single cells from each cell line. As can be seen from Table 4 below, the predictive model performed well since only CELL LINE 009 was not correctly predicted. The two plots in Part (a) and Part (b) of FIG. 7C further show the clustering of cells according to the disease state. It can be seen that healthy cells are clearly separated from diseased cells.

TABLE 4
Broken down of the cell-level performance of the predictions
for the predictive model trained using synthetic
pools but testing on single cell value.

| Cell line # | Pred | True | Predictions |
|---|---|---|---|
| CELL LINE 001 | 0.194320 | 0.0 | 0.193617 |
| CELL LINE 002 | 0.253787 | 0.0 | 0.251341 |
| CELL LINE 003 | 0.308828 | 0.0 | 0.308466 |
| CELL LINE 004 | 0.344662 | 0.0 | 0.343713 |
| CELL LINE 005 | 0.381917 | 0.0 | 0.381953 |
| CELL LINE 007 | 0.391825 | 0.0 | 0.392959 |
| CELL LINE 006 | 0.392930 | 0.0 | 0.393972 |
| CELL LINE 008 | 0.426124 | 0.0 | 0.426001 |
| CELL LINE 009 | 0.437766 | 1.0 | 0.438675 |
| CELL LINE 010 | 0.586013 | 1.0 | 0.585360 |
| CELL LINE 011 | 0.593789 | 1.0 | 0.593757 |
| CELL LINE 012 | 0.593848 | 1.0 | 0.594463 |
| CELL LINE 014 | 0.624509 | 1.0 | 0.623967 |
| CELL LINE 013 | 0.642103 | 1.0 | 0.640679 |

FIG. 7D illustrates another example performance of the predictive model trained and tested by pooling the morphological profiles using fixed feature vector. Briefly, only a predefined set of feature vectors were used for training and testing. The predefined set of feature vectors were purposely selected based on the features associated with the disease (e.g., they are considered disease-specific based on the previous studies). By selecting the fixed feature vectors in training the predictive model, the noise from the irrelevant features was masked. To train and test the predictive model, a dataset was divided in half by pooling half of the cell lines together (by disease state) for training, the test on the other half. The task was performed on 9 different combinations of all cell lines.

Table 5 shows the performance of the predicative model trained and tested on 9 different combinations of cell lines pooled together for training and testing using the fixed feature vectors. From the table, it can be seen that the predictive model performed well for all 9 combinations, with AUC values ranging from 0.944694 to 1.0. Part (a) and Part (b) of FIG. 7D show the PCA and TSNe reports for the ½ test/train split, respectively. The results in the PAC and TENs reports further confirmed the excellent performance of the predictive model trained and tested based on the synthetic pools and using fixed feature vectors.

TABLE 5
Performance of the predicative model trained and tested on
9 different combinations of cell lines pooled together for
training and testing using the fixed feature vectors.

| Group | AUC | Accuracy |
|---|---|---|
| 1 | 1.000000 | 0.935714 |
| 2 | 0.944684 | 0.621429 |
| 3 | 0.992857 | 0.500000 |
| 4 | 1.000000 | 0.500000 |
| 5 | 1.000000 | 0.964286 |
| 6 | 1.000000 | 1.000000 |
| 7 | 1.000000 | 0.750000 |
| 8 | 1.000000 | 0.992857 |
| 9 | 1.000000 | 0.650000 |

Overall, the above FIGS. 7A-7D show excellent performance of the predictive model that was trained by using the synthetically pooling of different cell lines from different donors. The predictive model trained in such a way performed well in predicting cell lines either for single cells or at well level, either pooled or not pooled. Accordingly, the predictive model trained using the synthetic pools can be an excellent tool in differentiating cells in healthy state and INAD disease state.

It is to be understood that while the predictive model was described with reference to the INAD disease, the predictive model similarly trained can be used in many different diseases, including neurodegenerative diseases or any other disease. That is, by using synthetic pools for training the predictive model, the noise caused by donor-specific variations can be minimized or eliminated, resulting in the improved performance of the predictive model in predicting disease state of cells with a unknown disease state.

### Example 3: Improved Performance of Predictive Models Trained with Synthetic Pools when Compared to Predictive Models Trained without a Synthetic Pool

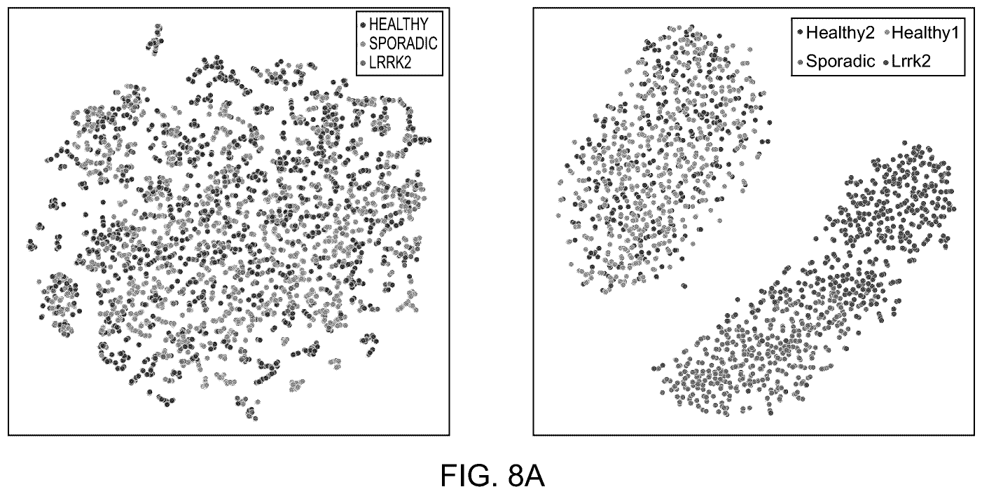

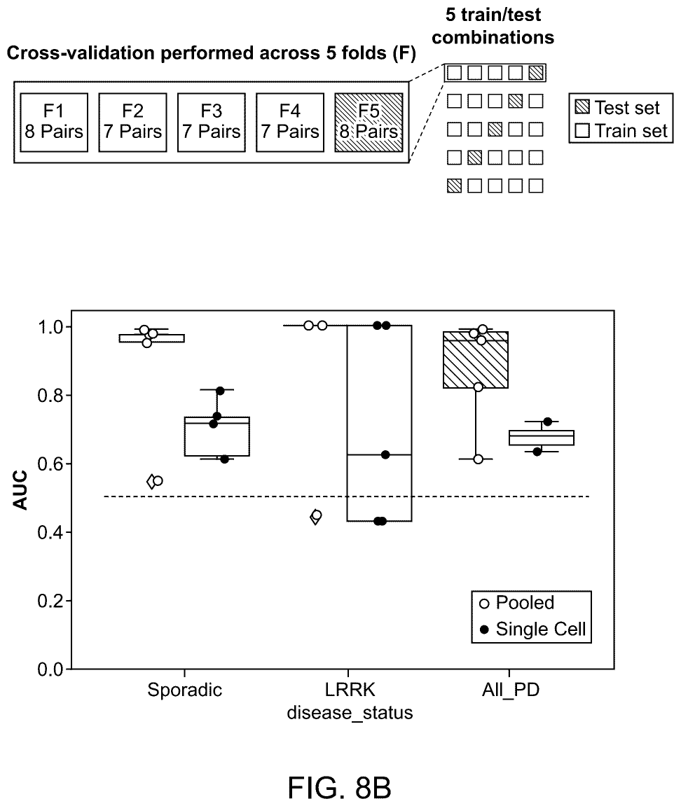

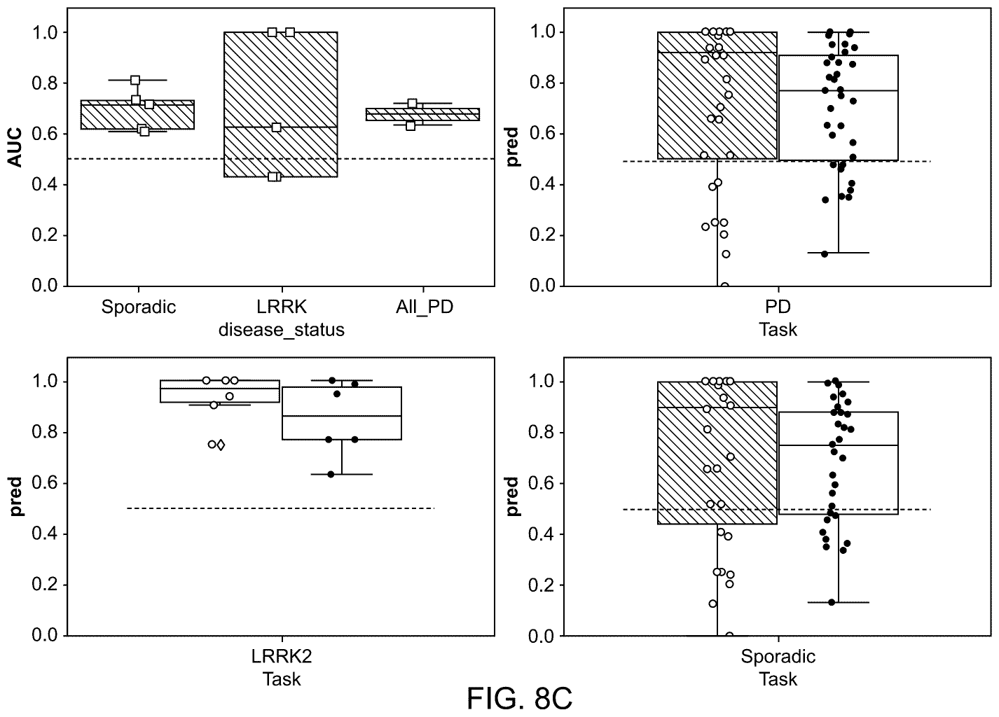

The advantages of using a synthetic pool in predictive model training were further confirmed by comparing the performance of a predictive model trained with or without a synthetic pool, as further described below with reference to FIGS. 8A-8D.

FIG. 8A depicts a performance comparison of a predictive model trained with or without a synthetic pool and tested at the well level using PD cell lines. Part (a) of the figure illustrates the well-level TSNe plot based on a testing by the predictive model trained using single cells without synthetically pooling the cell lines from different donors. The predictive model was trained and tested using the healthy cell lines and cell lines from PD sporadic subtype or LRRK2 subtype without a synthetic pool. The PD sporadic subtype and LRRK2 subtype were separately used for the training. The testing was performed at the well level by using mean well values. From the TSNe data in Part (a) of FIG. 8A, it can be seen that there is no evident cluster around healthy and disease states for the cell lines from healthy, sporadic subtype and LRRK2 subtype.

Part (b) of FIG. 8A illustrates the well-level TSNe plot based on a testing by the predictive model trained using synthetically pooled cell lines from different donors. The same dataset used to train the predictive model in Part (a) of FIG. 8A was used here, but different from Part (a), these cell lines were synthetically pooled together for the training process. The PD sporadic subtype and LRRK2 subtype were separately used for the training process. The testing was also performed at the well level by using mean well values. From the TSNe data in Part (b) of FIG. 8A, it can be seen that there is a clear PD phenotype that extends beyond donor-to-donor variation, and there is certain similarity in both sporadic and LRRK2 types when compared to healthy cell lines as the two subtypes are clustered close to each other while being separated from the healthy cells.

FIG. 8B depicts another performance comparison of a predictive model trained with or without a synthetic pool and tested at the cell-line level using PD cell lines. The dataset used for training and testing the predictive model was divided into 5 cross-validation folds, as illustrated in Part (a) of FIG. 8B. For the predictive model trained without using a synthetical pool, the four folds in each of the 5 train/test combinations were used as single cells at well level for training the predictive model, and the held out fold in the corresponding combination was used for testing. On the other hand, when the predictive model was trained with a synthetic pool, the four folds in each of the 5 train/test combinations were synthetically pooled together for training the predictive model and the held out fold in the corresponding combination was used for testing. The testing for both models trained with or without the synthetic pool was performed at individual wells and averaged at the cell level.

Part (b) of FIG. 8B shows the cell-line level AUC from the testing by the predictive model trained with or without the synthetic pool. In the plot, the box plots with orange dots represent the AUC values from the testing at the well level by the model trained without a synthetic pool, while the box plots with blue dots represent the AUC values from the testing at the well level by the model trained with a synthetic pool. As can be seen from the plot, the AUC values without using synthetic pools were around 0.7. This indicates that without synthetic pooling, the predictive model exhibits an acceptable predictive capacity. Additionally, the AUC values are much higher in the latter case (i.e., model trained with the synthetic pool), proving further confirmation of the improved performance of the model trained with the synthetic pool. The plot also provided evidence that there is a clear and detectable phenotype of PD in the tested cell lines (e.g., fibroblasts). It is to be understood that in the plot, “All_PD” means that the different subtypes of PD (e.g., sporadic and LRRK2) were mixed together as a general PD population during the training process and testing process, while “Sporadic” and “LRRK” means that the two subtypes were separately trained and tested.

FIG. 8C depicts another performance comparison of a predictive model trained with or without a synthetic pool and tested at the cell-line level using PD cell lines. Part (a) of FIG. 8C illustrates the performance of the predictive model trained using cell lines without a synthetic pool. The training and testing of the predictive model were performed in a cross-validation fashion (e.g., through a train/test split) at the cell line level. The testing results are shown in the plot at the well level and the AUC values for each healthy/PD pair were displayed, including the healthy/sporadic pair, healthy/LRRK2 pair, and healthy/all PD pair, where the “All PD” means that the two subtypes included in the dataset were pooled together. As can be seen from the box plots in Part (a) of FIG. 8C, the AUC values for the three pairs were between 0.6-0.7 range when the predictive model was trained without using a synthetic pool. This indicates that even a predictive model trained without using a synthetic pool exhibits acceptable predictive capacity.

Parts (b)-(d) in FIG. 8C further illustrate the performance of the predictive model trained using cell lines with a synthetic pool. The training and testing of the predictive model were also performed in a cross-validation fashion at the cell line level but with a synthetic pool during the model training process. The testing results are shown at the well level in the three plots shown in Parts (b)-(d) for each healthy/PD pair, including the healthy/all PD pair, healthy/LRRK2 pair, and healthy/sporadic pair, respectively. The box plots with blue dots in each plot correspond to the predictive model trained using the pooled data from all PD data (i.e., cells of sporadic and LRRK2 subtypes are mixed), while the box plots with orange dots in each plot correspond to the predictive model trained using the pooled sporadic subtype and pooled LRRK2 subtype, separately. From the three plots, it can be seen the predictive models trained with synthetic pools (either all PD pooled together or each subtype separately pooled) generally have higher AUC values (e.g., between 0.7-1.0) when compared to the AUC values in Part (a) of FIG. 8C. In addition, the decrease of the AUC values for the box plots with orange color proves that both the LRRK2- and the sporadic-specific phenotypes contribute to building a stronger model, suggesting that there is a phenotype of PD specifically in the tested cell lines (e.g., fibroblasts).

FIG. 8D depicts another performance comparison of a predictive model trained with or without a synthetic pool and tested at cell-line level using INAD cell lines. The dataset used for training and testing the predictive model was divided according to a 50% train/test split, and the results were shown in the plot in FIG. 8D. In the plot, the box plot with blue dots corresponds to the “pooled” data, which means that the training data was synthetically pooled. The testing was performed at the sing cell-line level. For the box plot with orange dots that correspond to the “un-pooled” data, the training was performed on the regularly averaged wells (e.g., mean well values) without synthetically pooling different cell lines.

From the AUC values in the plot in FIG. 8D, it can be seen that the all-cell-lines pooling method (i.e., synthetically pooling different cell lines in the model training process) is much more powerful. This is especially true considering that there were only few cell lines (e.g., just 4 cell lines) available and there were very few samples for an effective smoothing of donor-to-donor variation during the training and testing processes. The results from the plot in FIG. 8D indicate that the predictive model trained with the synthetic pool from the limited number of cell lines performed better than using other classical models trained without a synthetic pool.

Overall, the above FIGS. 8A-8D further show improved performance of the predictive model trained with a synthetic pool when compared to a predictive model trained without a synthetic pool. The improved performance was confirmed by using different diseases and/or subtypes of diseases, which further supports that a predictive model trained with a synthetic pool can be applied to many different diseases or disease subtypes. In addition, the results also show that a predictive model trained with a synthetic pool can be an effective tool when there are limited numbers of cell lines and/or limited numbers of samples available for disease prediction.

### Example 4: Successfully Identifying Phenotypes of Specific Diseases

In various embodiments, synthetic pools when used for training and testing the predictive model also allow to better identify disease-specific features, due to its mask of donor-specific variations that generally hide the features characterizing a disease. To identify the features specific to a disease (e.g., INAD disease), synthetic pools are created and used for both training and testing of a predictive model for characterizing the disease. In one example, 9 50% cross-validation folds were created and used to train and test the predictive model for the INAD disease. After the training and testing of the predictive model, cells with the perfect prediction scores (e.g., AUC 1.0) were then selected and analyzed for identifying features specific to the disease. After analysis, certain features recurred in all 9 folds, which were considered the top-ranked features.

To limit the number of features specific to the INAD disease for easier characterization, the selected top-ranked features were further filtered to remove correlated features, that is, to remove features that correlated with each other. Filtering the correlated features may remove redundancy in characterizing a disease since these features may carry the same information or provide duplicated information since the information from one feature can generally tell the information for another correlated feature. After filtration to remove the redundancy of the correlated features, the number of features for characterizing a disease can be further decreased. For example, for the above example of the INAD disease, the total number of features decreased from 250 to 55 after filtration.

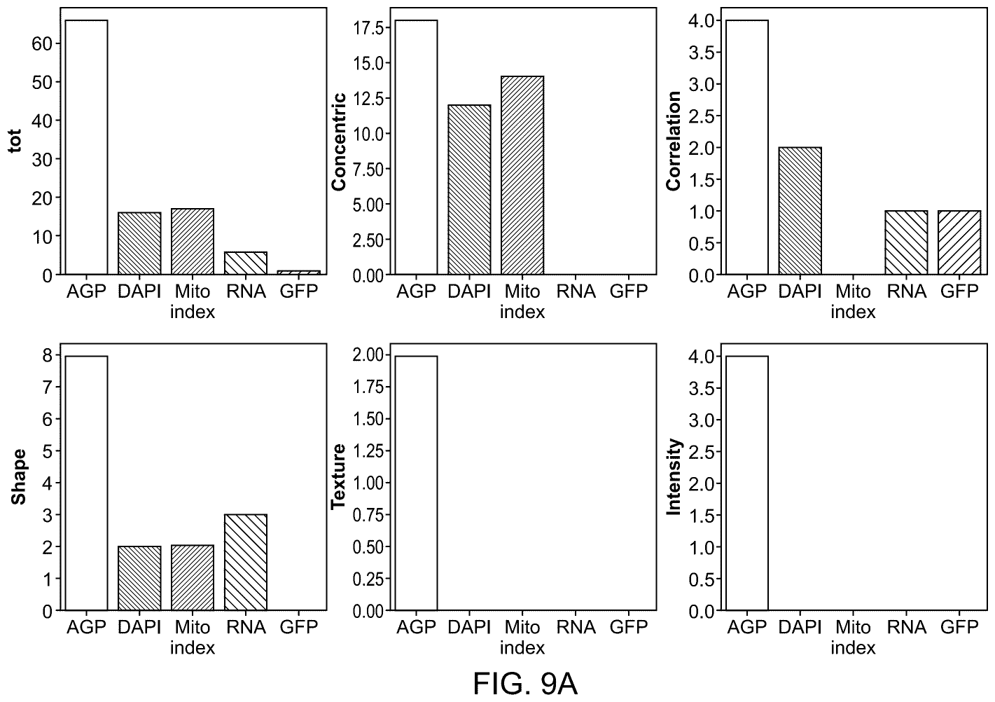

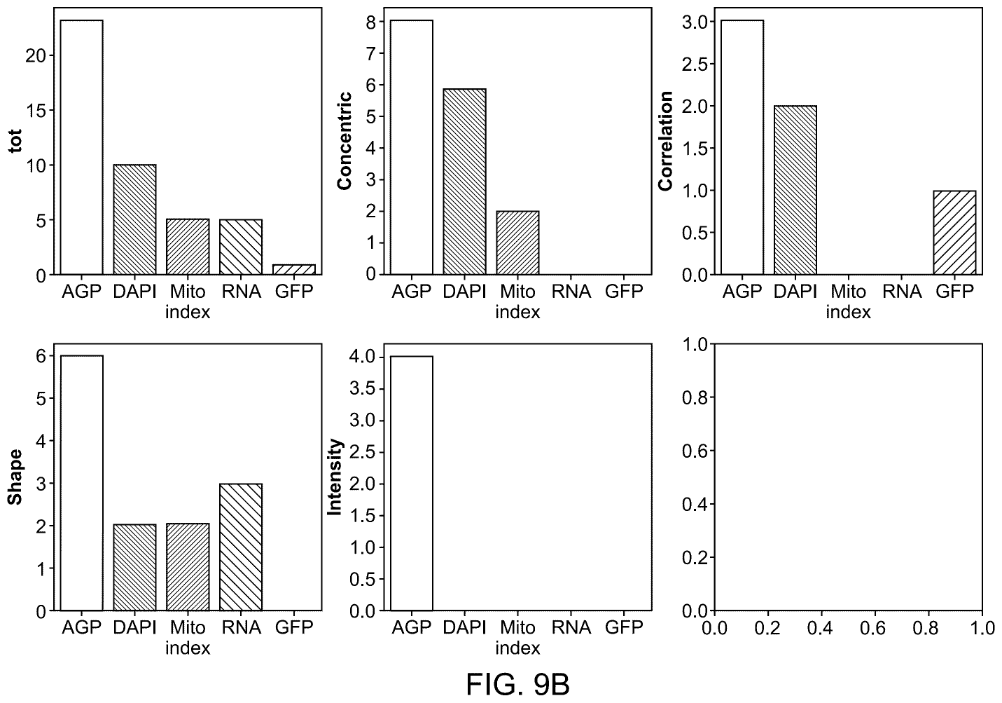

FIGS. 9A and 9B illustrate plots for presenting the occurrence of top-ranked features in detection channels from different aspects. The data were summarized based on the information used for training and testing the predictive model. FIG. 9A illustrates plots for presenting the occurrence of top-ranked features in detection channels from different aspects before filtration, and FIG. 9B illustrates plots for presenting the occurrence of top-ranked features in detection channels from different aspects after filtration. As can be seen from FIGS. 9A and 9B, after the filtration to remove certain correlated features, the total number of features for characterizing the INAD disease is greatly reduced.

In the plots in FIGS. 9A and 9B, the top-ranked features were further summarized from different aspects. In both figures, the “tot” represents the total number of top-ranked features occurring within each channel. For example, for the left plot in FIG. 9B, the number of top-ranked features occurred in the AGP channel 23 times, 10 times in the DAPI channel, 5 times in the Mito channel, 5 times in the RNA channel, and once in the GFP channel. “Concentric” measures certain features with increasing diameters. This means that features with increasing diameters occurred 8 times in the AGP channel, 6 times in the DAPI channel, and 2 times in the Mito channel. “Correlation” basically measures how much a channel is correlated to another. There are many different ways to determine correlations between channels. The number of each channel still means the occurrence in each channel. “Shape” measures the occurrence of certain shape-related features in each channel. “Texture” measures the occurrence of certain texture-related features in each channel. “Intensity” measures the occurrence of certain intensity-related features in each channel. Basically, the plots measured the importance of features in terms of occurrence.

In various embodiments, after the top-ranked features were determined for the disease (e.g., the INAD disease in the above example), these features may be considered as disease-specific. These disease-specific features can be used to highlight the phenotype of the disease, for example, for later detection of the disease, among other applications. It is to be understood that while the INAD disease was described in identifying the disease-specific features, the above descriptions are not limited to the INAD disease, but rather can be applied to identify disease-specific features for any other disease.

1-122. (canceled)

123. A method comprising:
obtaining or having obtained one or more cells of a common state;
capturing a plurality of images corresponding to the one or more cells; and
analyzing the plurality of images using a predictive model to predict a presence or absence of a known disease state for the one or more cells, the predictive model trained to distinguish between morphological profiles of healthy cells and cells in a known disease state,
wherein the predictive model is trained using training data generated from at least one cohort of synthetically pooled cells of the known disease state.

124. The method of claim 123, wherein:
the at least one cohort of synthetically pooled cells are combined from a plurality of sources, which causes source-specific variations to be smoothened and state-specific features to be highlighted when training the predictive model,
the at least one cohort of synthetically pooled cells is built by randomly selecting a number of single cells or randomly selecting a number of tiles,
the synthetically pooled cells are formed by pooling together a plurality of cell lines of the known disease state or healthy state, wherein pooling together the plurality of cell lines comprises combining embeddings or fixed feature vectors of randomly selected single cells without physically pooling together the randomly selected single cells, and the combining comprises averaging the embeddings or fixed feature vectors of the randomly selected single cells, or
the plurality of cell lines are obtained from different subjects of the known disease state or healthy state.

125. The method of claim 123, wherein the predictive model trained to distinguish between the morphological profiles of healthy cells and cells in the known disease state achieves an AUC of at least 0.95 or an accuracy of at least 0.88.

126. The method of claim 123, wherein the predictive model is trained by:
capturing a plurality of images corresponding to the randomly selected single cells of the known disease state or healthy state; and
using the plurality of images corresponding to the randomly selected single cells of the known disease state or healthy state to train the predictive model to distinguish between the morphological profiles of cells of the known disease state and cells of the healthy state,
wherein using the plurality of images corresponding to the randomly selected single cells of the known disease state or healthy state to train the predictive model further comprises averaging embeddings of the plurality of images corresponding to the randomly selected single cells of the known disease state or healthy state.

127. The method of claim 123, wherein:
the one or more cells of a common state comprise cells of a single cell line from a single subject,
the predictive model is trained to predict the presence or absence of the known disease state with a prediction probability, or
the healthy cells or the cells in the known disease state serve as a reference ground truth for training the predictive model.

128. The method of claim 123, wherein, to distinguish between the morphological profiles of healthy cells and cells in the known disease state for the one or more cells of a common state, the predictive model is trained to compare an averaged embedding of the one or more cells of a common state to an averaged embedding of the plurality of images corresponding to the randomly selected single cells of the known disease state or healthy state.

129. The method of claim 123, further comprising:
prior to capturing the plurality of images corresponding to the one or more cells of a common state, providing a perturbation to the one or more cells of a common state, the perturbation causing the one or more cells from a known disease state to an unknown disease state;
subsequent to analyzing the plurality of images of the one or more cells of a common state, comparing the predicted state of the one or more cells to the known disease state of the one or more cells known before providing the perturbation; and
based on the comparison, identifying the perturbation as having one of a therapeutic effect, a detrimental effect, or no effect.

130. The method of claim 123, wherein:
the predictive model is one of a neural network, random forest, or regression model.

131. The method of claim 123, wherein:
each of the morphological profiles comprises values of imaging features or comprise a transformed representation of images that define a known disease state or a healthy state of a cell.

132. The method of claim 123, wherein each cell in the one or more cells of a common state is one of a stem cell, a partially differentiated cell, or a terminally differentiated cell.

133. The method of claim 123, wherein each cell in the one or more cells of a common state is a somatic cell selected from a fibroblast or a peripheral blood mononuclear cell (PBMC).

134. The method of claim 123, wherein the one or more cells of a common state are obtained from a subject through a tissue biopsy or blood draw.

135. The method of claim 123, wherein the morphological profile is extracted from a layer of a penultimate deep learning neural network.

136. The method of claim 123, further comprising:
prior to capturing the plurality of images corresponding to the one or more cells of a common state, staining or having stained the one or more cells of a common state using one or more fluorescent dyes.

137. The method of claim 136, wherein:
at least 5 or 30 cell features derive from fluorescently labeled biomarkers identifying plasma membrane,
at least 5 or 25 cell features derive from fluorescently labeled biomarkers identifying cell nucleus,
at least 5 or 10 cell features derive from fluorescently labeled biomarkers identifying endoplasmic reticulum,
at least 5 or 35 cell features derive from fluorescently labeled biomarkers identifying mitochondria,
at least 5 or 10 cell features derive from fluorescently labeled biomarkers identifying RNA, or
at least 20 or 60 correlated cell features derive from various fluorescence channels.

138. The method of claim 123, wherein:
each of the plurality of images corresponding to the one or more cells of a common state corresponds to a fluorescent channel, and
the steps of obtaining or having obtained the one or more cells of a common state and capturing the plurality of images corresponding to the one or more cells of a common state are performed in a high-throughput format using an automated array.

139. The method of claim 123, wherein:
a common state is one of a common disease state, a common source, a common processing state, or a common growth state,
the disease state of the cell predicted by the predictive model is a classification of at least two categories.

140. The method of claim 139, wherein the at least two categories comprise a presence or absence of a neurodegenerative disease, and the neurodegenerative disease is any one of Parkinson's Disease (PD), Alzheimer's Disease, Amyotrophic Lateral Sclerosis (ALS), Infantile Neuroaxonal Dystrophy (INAD), Multiple Sclerosis (MS), Amyotrophic Lateral Sclerosis (ALS), Batten Disease, Charcot-Marie-Tooth Disease (CMT), Autism, post-traumatic stress disorder (PTSD), schizophrenia, frontotemporal dementia (FTD), multiple system atrophy (MSA), and a synucleinopathy.

141. The method of claim 123, further comprising:
identifying a plurality of features associated with the known disease state when the one or more cells are predicted to be the known disease state;
ranking the plurality of features according to a degree of difference of the features between the known disease state and the healthy state;
selecting a list of top-ranked features according to a predefined threshold;
filtering the top-ranked features by removing a subset of features that are correlated; and
updating the list of top-ranked features by excluding the subset of features, wherein the updated list of top-ranked features are designated as a phenotype for characterizing the known disease state.

142. A non-transitory computer readable medium comprising instructions that, when executed by a processor, cause the processor to:
capture a plurality of images corresponding to one or more cells of a common state; and
analyze the plurality of images using a predictive model to predict a presence or absence of a known disease state for the one or more cells, the predictive model trained to distinguish between morphological profiles of healthy cells and cells in a known disease state,
wherein the predictive model is trained using training data generated from at least one cohort of synthetically pooled cells of the known disease state.
